(19) United States
(12) Patent Application Publication
(10) Pub. No.: US 2026/0125349 A1
(43) Pub. Date: May 7, 2026

(54) PROCESS FOR THE FLUORINATION AND/OR CYCLIZATION OF AN AMINO ALKENE OR ALKYNE IN A CONTINUOUS STREAM AND FACILITY FOR PERFORMING THE PROCESS

(71) Applicants: UNIVERSITE DE POITIERS, POITIERS CEDEX 9 (FR); CENTRE NATIONAL DE LA RECHERCHE SCIENTIFIQUE, PARIS (FR); SORBONNE UNIVERSITE, PARIS (FR)

(72) Inventors: ZAHRA ABADA, POITIERS (FR); AGNÈS MARTIN-MINGOT; YVES BLERIOT, POITIERS (FR); SÉBASTIEN THIBAUDEAU, DIENNE (FR); ALI ABOU-HASSAN, PARIS (FR)

(21) Appl. No.: 19/118,044

(22) PCT Filed: Oct. 3, 2023
(86) PCT No.: PCT/EP2023/077346

§ 371 Date: Apr. 3, 2025

(30) Foreign Application Priority Data
Oct. 4, 2022 (FR) FR2210129

(51) Int. Cl. C07D 241/04; C07C 209/00; C07C 311/16; C07D 209/08; C07D 211/14; C07D 211/82; C07D 279/02

(52) U.S. Cl. CPC C07D 241/04; C07C 209/00; C07C 311/16; C07D 209/08; C07D 211/14; C07D 211/82; C07D 279/02

(57) ABSTRACT

The present invention relates to a process for the fluorination and/or cyclization of an amino alkene or alkyne in a continuous-stream microreactor. The invention also relates to a facility for performing such a process.

### SUBJECT OF THE INVENTION

The present invention relates to a process for the fluorination and/or cyclization of an amino alkene or alkyne in a continuous-stream microreactor. The invention also relates to a facility for performing such a process.

### BACKGROUND OF THE INVENTION

In a superacid medium, H₀ acidity values are lower than −12, and as such all molecules, even simple alkanes, react as bases. Superacid systems are therefore of great interest because they give access to a particular type of reactivity involving polyprotonated molecules. Numerous processes have been developed in a superacid medium in recent years, and may be envisaged for both laboratory-scale and industrial-scale synthesis.

For example, Michelet et al. describes processes for fluorinating amino alkenes or alkynes using the superacid HF/SbF₅ (J. Fluorine Chem. 2018, 214, 68-79). This transformation is most particularly interesting because it allows access to fluoro amine units, which are sought after in medicinal chemistry. Moreover, WO 95/03312 shows that vinorelbine can be converted into the anticancer agent Javlor® via a gem-difluorination reaction in the presence of the superacid HF/SbF₅.

However, these systems have a number of drawbacks that limit their use. In particular, the reagents that generate a superacid medium, such as HF, are toxic, corrosive and hazardous to handle. In addition, many solvents and nucleophiles are not compatible with these media. Finally, the high reactivity of these systems does not make it possible to control the outcome of the reactions: cascade reactions and polyfluorination may be observed.

There is thus a real need to develop a process that facilitates the handling of superacids and allows better control of their reactivity, most particularly when they are placed in contact with an amino alkyne or alkene.

In this context, the Applicant has shown that the use of microfluidics, and more specifically of a continuous-flow microreactor, allows these limitations to be overcome. More specifically, it has been shown, surprisingly, that the use of a continuous-flow microreactor allows the preparation of fluorinated or cyclized compounds, which are difficult to access or even inaccessible via an equivalent reaction under static (or “batch”) conditions. It has also been shown that the productivity of these reactions under flow conditions was much higher than that obtained under static conditions.

### SUMMARY OF THE INVENTION

The invention thus relates to a process for the fluorination and/or cyclization of an amino alkene or alkyne, involving:
a) providing a first phase comprising an amino alkene or alkyne and a second phase comprising a superacid reagent,
b) placing the first and second phases in contact in a continuous-flow microreactor, and
c) recovering the fluorination and/or cyclization product of said amino alkene or alkyne.

In a particular embodiment, the residence time of the first phase and the second phase in the continuous-flow microreactor in step (b) is between 2 seconds and 400 seconds.

In a particular embodiment, the flow rate of the first phase and the flow rate of the second phase in step (b) are independently between 0.1 mL/min and 3.5 mL/min, for example between 0.25 m/min and 3.0 mL/min.

In a particular embodiment, the continuous-flow microreactor comprises a micro-mixer and a tubular pipe, in which the tubular pipe preferably has:
a length of between 20 cm and 800 cm, and
an inside diameter of between 0.5 mm and 2.5 mm, preferably between 0.5 mm and 1.5 mm, and better still between 0.7 mm and 1.2 mm.

In a particular embodiment, the placing in contact in the continuous-flow microreactor is performed at a temperature of between −70° C. and 25° C.

In a particular embodiment, the process according to the invention is performed in a facility comprising:
a storage unit (1) for a first phase comprising an amino alkene or alkyne,
a storage unit (1′) for a second phase comprising a superacid reagent,
a first phase continuous feed means (3) connected to the first phase storage unit (1), and to a first phase equilibration tubular pipe (4),
a second phase continuous feed means (3′) connected to the second phase storage unit (1′) and to a second phase equilibration tubular pipe (4′),
a continuous-flow microreactor (2) comprising a micro-mixer (21) comprising two inlets and one outlet, and a tubular pipe (22) comprising one inlet and one outlet, in which the inlet of the tubular pipe (22) is connected to the outlet of the micro-mixer (21),
   in which the first phase equilibration tubular pipe (4) is connected to the first inlet of the micro-mixer (21) and the second phase equilibration tubular pipe (4′) is connected to the second inlet of the micro-mixer (21), and
a collection unit (5), connected to the outlet of the tubular pipe (22) of the microreactor (2).

In a particular embodiment, the superacid reagent is chosen from HF/MF₅ and HSO₃F/MF₅, where M is Sb, As, P, Ta, or Nb; preferably the superacid reagent is HF/SbF₅.

In a particular embodiment, the first phase is a solution of said amino alkene or alkyne in HF, preferably in a concentration of between 0.5 and 1.0 mol/L.

Said amino alkene or alkyne may in particular be an allyl or propargyl amine. In a particular embodiment, said amino alkene or alkyne is an amino alkene of formula (Ia):

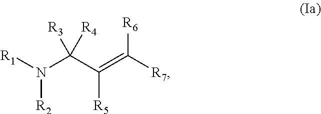

in which:
R₁, R₂, R₃, R₄, R₅, R₆ and R₇ are independently chosen from hydrogen, halogen, C₁-C₁₂ alkyl, C₂-C₁₂ alkenyl, C₂-C₁₂ alkynyl, C₁-C₁₂ heteroalkyl, C₃-C₁₂ cycloalkyl, C₂-C₁₂ heterocycloalkyl, aryl, heteroaryl, a group —S(O)₂—R₈ and a group —C(O)—R₉, where R₈ and R₉ are chosen independently from C₁-C₁₂ alkyl, C₂-C₁₂ alkenyl, C₂-C₁₂ alkynyl, C₁-C₁₂ heteroalkyl, C₃-C₁₂ cycloalkyl, C₂-C₁₂ heterocycloalkyl, aryl, and heteroaryl,
said alkyl, alkenyl, alkynyl, heteroalkyl, cycloalkyl, heterocycloalkyl, aryl and heteroaryl groups being optionally substituted, or
two groups each chosen from R₁, R₂, R₃, R₄, R₅, R₆ and R₇ may alternatively form, together with the atom(s) to which they are attached, an optionally substituted 3- to 12-membered ring.

In particular, the amino alkene of formula (Ia) may be such that:
R₁ and R₂ are chosen independently from a hydrogen, a C₂-C₁₂ alkenyl, an aryl [optionally substituted with a C₁-C₆ alkyl, a nitro, or a fluoro group chosen from —CF₃, —OCF₃, —SCF₃, —OCF₂R′, —OCF(R′)₂, —SCF₂R′ and —SCF(R′)₂ where each R′ independently represents a hydrogen, a halogen, a C₁-C₆ alkyl, a C₂-C₆ alkenyl, a C₂-C₆ alkynyl or a C₃-C₆ cycloalkyl], —S(O)₂—R₈ and —C(O)—R₉, where R₈ and R₉ are independently aryl optionally substituted with C₁-C₆ alkyl, nitro or a fluoro group chosen from —CF₃, —OCF₃, —SCF₃, —OCF₂R′, —OCF(R′)₂, —SCF₂R′ and —SCF(R′)₂ where each R′ independently represents hydrogen, halogen, C₁-C₆ alkyl, C₂-C₆ alkenyl, C₂-C₆ alkynyl or a C₃-C₆ cycloalkyl,
   or alternatively R₁ and R₂, together with the atom to which they are attached, form a piperidine or piperazine, optionally substituted with an aryl or an acetyl,
R₃ and R₄ are hydrogens, and
R₅, R₆ and R₇ are independently chosen from hydrogen and halogen (preferably at least two from among R₅, R₆ and R₇ are hydrogen).

In another particular embodiment, said amino alkene or alkyne is an amino alkyne of formula (Ib):

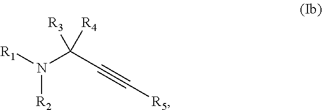

in which:
R₁, R₂, R₃, R₄ and R₅ are independently chosen from hydrogen, halogen, C₁-C₁₂ alkyl, C₂-C₁₂ alkenyl, C₂-C₁₂ alkynyl, C₁-C₁₂ heteroalkyl, C₃-C₁₂ cycloalkyl, C₂-C₁₂ heterocycloalkyl, aryl, heteroaryl, a group —S(O)₂—R₈ and a group —C(O)—R₉, where R₈ and R₉ are chosen independently from C₁-C₁₂ alkyl, C₂-C₁₂ alkenyl, C₂-C₁₂ alkynyl, C₁-C₁₂ heteroalkyl, C₃-C₁₂ cycloalkyl, C₂-C₁₂ heterocycloalkyl, aryl, and heteroaryl,
said alkyl, alkenyl, alkynyl, heteroalkyl, cycloalkyl, heterocycloalkyl, aryl and heteroaryl groups being optionally substituted, or
two groups each chosen from R₁, R₂, R₃, R₄ and R₅ may alternatively form, together with the atom(s) to which they are attached, an optionally substituted 3- to 12-membered ring.

In particular, the amino alkene of formula (Ib) may be such that:
R₁ and R₂ are chosen independently from a hydrogen, a C₂-C₁₂ alkenyl, an aryl [optionally substituted with a C₁-C₆ alkyl, a nitro, or a fluoro group chosen from —CF₃, —OCF₃, —SCF₃, —OCF₂R′, —OCF(R′)₂, —SCF₂R′ and —SCF(R′)₂ where each R′ independently represents a hydrogen, a halogen, a C₁-C₆ alkyl, a C₂-C₆ alkenyl, a C₂-C₆ alkynyl or a C₃-C₆ cycloalkyl], —S(O)₂—R₈ and —C(O)—R₉, where R₈ and R₉ are independently aryl optionally substituted with C₁-C₆ alkyl, nitro or a fluoro group chosen from —CF₃, —OCF₃, —SCF₃, —OCF₂R′, —OCF(R′)₂, —SCF₂R′ and —SCF(R′)₂ where each R′ independently represents hydrogen, halogen, C₁-C₆ alkyl, C₂-C₆ alkenyl, C₂-C₆ alkynyl or a C₃-C₆ cycloalkyl,
   or alternatively R₁ and R₂, together with the atom to which they are attached, form a piperidine or piperazine, optionally substituted with an aryl or an acetyl, and
R₃, R₄ and R₅ are hydrogens.

In a preferred mode, said amino alkene or alkyne is chosen from:

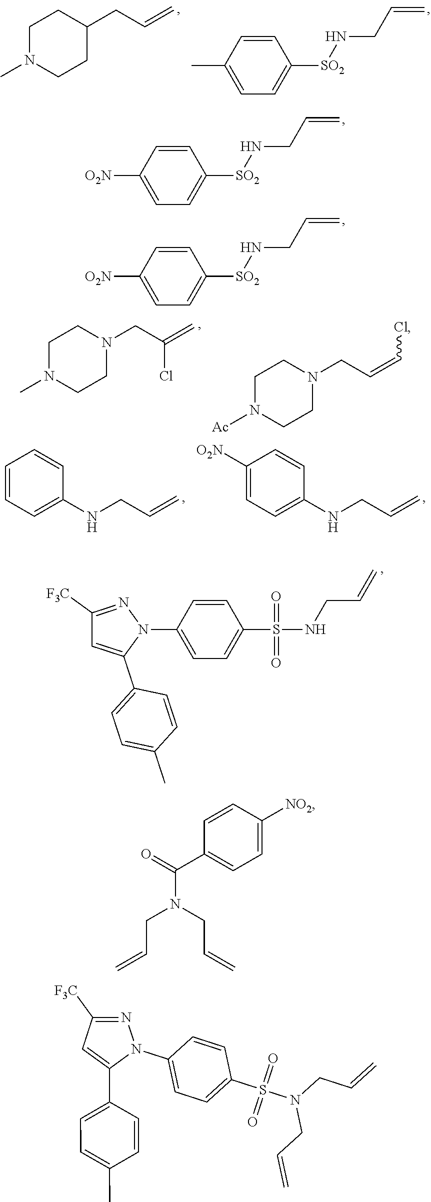

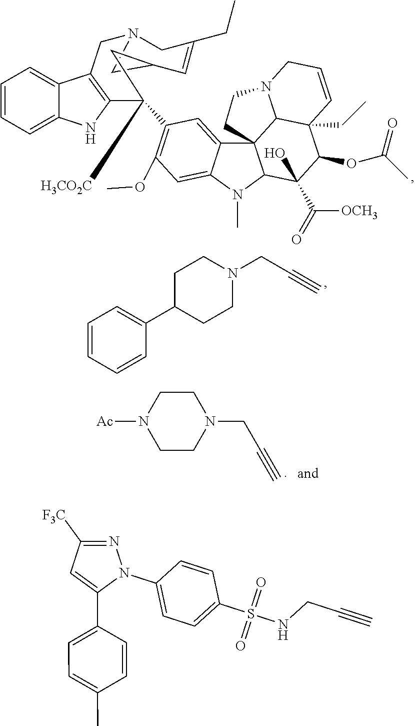

In one embodiment, said amino alkene or alkyne is chosen from:

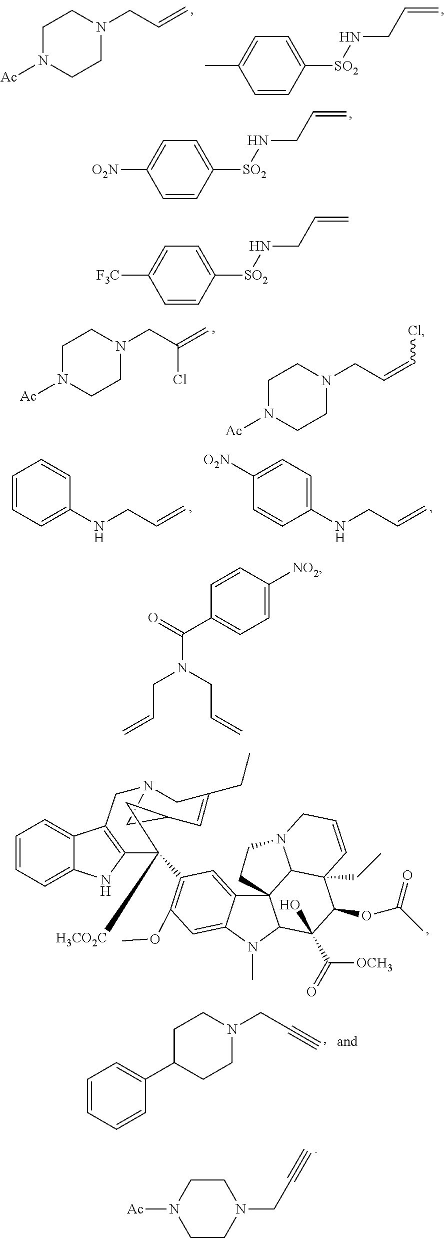

The invention also relates to a facility for performing the process as defined in the present patent application, characterized in that it comprises:
a storage unit (1) for a first phase comprising an amino alkene or alkyne,
a storage unit (1′) for a second phase comprising a superacid reagent,
a first phase continuous feed means (3) connected to the first phase storage unit (1), and to a first phase equilibration tubular pipe (4),
a second phase continuous feed means (3′) connected to the second phase storage unit (1′) and to a second phase equilibration tubular pipe (4′),
a continuous-flow microreactor (2) comprising a micro-mixer (21) comprising two inlets and one outlet, and a tubular pipe (22) comprising one inlet and one outlet, in which the inlet of the tubular pipe (22) is connected to the outlet of the micro-mixer (21),
   in which the first phase equilibration tubular pipe (4) is connected to the first inlet of the micro-mixer (21) and the second phase equilibration tubular pipe (4′) is connected to the second inlet of the micro-mixer (21), and
a collection unit (5), connected to the outlet of the tubular pipe (22) of the microreactor (2).

The tubular pipe (22) of the continuous-flow microreactor (2) preferably has:
a length of between 20 cm and 800 cm, and
an inside diameter of between 0.5 and 2.5 mm, and preferably between 0.7 and 1.2 mm. Preferably, said facility also comprises:
a 3-way valve (6) providing the connection between the first phase continuous feed means (3), the first phase storage unit (1), and the first phase equilibration tubular pipe (4), and/or
a 3-way valve (6′) providing the connection between the second phase continuous feed means (3′), the second phase storage unit (1′) and the second phase equilibration tubular pipe (4′).

### FIGURES

FIG. 1 is a diagram illustrating the process according to one embodiment of the invention, and also a particular schematic facility for performing such a process.

FIG. 2 is a diagram illustrating the process according to one embodiment of the invention, and also a particular schematic facility for performing such a process, also comprising washing units.

### DETAILED DESCRIPTION

### Definitions

Unless otherwise indicated, when a range is expressed using the expression “between”, the limit values are included within the range described.

The term “alkyl” means a saturated, linear or branched aliphatic hydrocarbon-based group. A “C₁-C₁₂ alkyl” is an alkyl containing from 1 to 12 carbon atoms. Examples of alkyl (or C₁-C₁₂ alkyl) are notably methyl, ethyl, propyl, isopropyl, butyl, isobutyl, tert-butyl, pentyl, hexyl, heptyl, octyl, nonyl, decyl, undecyl or dodecyl. Preferably, the C₁-C₁₂ alkyl is a C₁-C₆ alkyl (for example: methyl, ethyl, propyl, isopropyl, butyl, isobutyl, tert-butyl, pentyl or hexyl).

The term “alkenyl” means a linear or branched, unsaturated aliphatic hydrocarbon-based group comprising at least one carbon-carbon double bond. A “C₂-C₁₂ alkenyl” is an alkenyl containing from 2 to 12 carbon atoms. Examples of alkenyl (or C₂-C₁₂ alkenyl) are notably ethenyl, propenyl, isopropenyl, butenyl, isobutenyl, pentenyl, hexenyl, heptenyl, octenyl, nonenyl, decenyl, undecenyl or dodecenyl. Preferably, the C₂-C₁₂ alkenyl is a C₂-C₆ alkenyl (for example: ethenyl, propenyl, isopropenyl, butenyl, isobutenyl, pentenyl, or hexenyl).

The term “alkynyl” means a linear or branched, unsaturated aliphatic hydrocarbon-based group comprising at least one carbon-carbon triple bond. A “C₂-C₁₂ alkynyl” is an alkynyl containing from 2 to 12 carbon atoms. Examples of alkynyl (or C₂-C₁₂ alkynyl) are notably ethynyl, propynyl, butynyl, pentynyl, hexynyl, heptynyl, octynyl, nonynyl, decynyl, undecynyl or dodecynyl. Preferably, the C₂-C₁₂ alkynyl is a C₂-C₆ alkynyl (for example: ethynyl, propynyl, butynyl, pentynyl or hexynyl).

The term “heteroalkyl” means an alkyl as defined above, in which the carbon chain comprises at one and/or other of its ends (notably the end attached to the rest of the molecule), and/or is interrupted with, at least one heteroatom, such as O, N, P, Se or S. Examples of heteroalkyl are notably alkoxy (—O-alkyl), alkylthio (—S-alkyl), alkylamino (—NH(alkyl) or —N(alkyl)₂), organophosphorus (—P(O)(alkyl)₂) and organoselenium (—Se(Alkyl)₂ or —Se(O)(alkyl)NR or —Se(O)(alkyl)₂). A “C₁-C₁₂ heteroalkyl” is a heteroalkyl containing from 1 to 12 carbon atoms. Preferably, a C₁-C₁₂ heteroalkyl is a C₁-C₆ heteroalkyl.

Examples of heteroalkyl (or C₁-C₁₂ or C₁-C₆ heteroalkyl) are notably methoxy, ethoxy, propyloxy, isopropyloxy, butyloxy, isobutyloxy, tert-butyloxy, pentyloxy, hexyloxy, methylthio, ethylthio, propylthio, isopropylthio, butylthio, isobutylthio, tert-butylthio, pentylthio, hexylthio, methylamino, ethylamino, propylamino, isopropylamino, butylamino, isobutylamino, tert-butylamino, pentylamino, hexylamino, methylthio, ethylthio, propylthio, isopropylthio, butylthio, isobutylthio, tert-butylthio, pentylthio, hexylthio, methylphospho, ethylphospho, propylphospho, isopropylphospho, butylphospho, isobutylphospho, tert-butylphospho, pentylphospho, hexylphospho, methylseleno, ethylseleno, propylseleno, isopropylseleno, butylseleno, isobutylseleno, tert-butylseleno, pentylseleno, or hexylseleno.

The term “cycloalkyl” means an optionally unsaturated (preferably saturated) aliphatic monocyclic or polycyclic hydrocarbon-based group which may be condensed, bridged and/or spiro-connected. A “C₃-C₁₂ cycloalkyl” is a cycloalkyl containing 3 to 12 carbon atoms. Examples of C₃-C₁₂ or C₃-C₆ cycloalkyl are notably cyclopropyl, cyclopentyl or cyclohexyl.

The term “heterocycloalkyl” means a cycloalkyl as defined above, also comprising at least one heteroatom, such as N, S, P, Se or O. A “C₂-C₁₂ heterocycloalkyl” is a cycloalkyl containing 2 to 12 carbon atoms and at least one heteroatom. Preferably, a C₂-C₁₂ heterocycloalkyl is preferably C₂-C₆ heterocycloalkyl. Examples of heterocycloalkyls are notably: 3-dioxolane, benzo-[1,3]-dioxolyl, azetidinyl, oxetanyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, oxozolinyl, oxazolidinyl, isoxazolinyl, isoxazolidinyl, thiazolinyl, thiazolidinyl, isothiazolinyl, isothiazolidinyl, dihydropyranyl, tetrahydropyranyl, tetrahydrofuranyl, 7-oxabicyclo[2,2,1]heptanyl, cycloalkylphosphine and tetrahydrothiophenyl.

The term “aryl” means a monocyclic or polycyclic aromatic carbocyclic group, preferably containing from 6 to 20 ring members. Examples of aryl groups are phenyl, biphenyl and naphthyl, preferably phenyl. The aryl group, in particular phenyl, is optionally substituted with, for example, one or more (preferably only one) groups chosen from C₁-C₆ alkyl (e.g. methyl), —NO₂, —CF₃, and other fluoro substituents such as —OCF₃, —SCF₃, —OCF₂R′, —OCF(R′)₂, —SCF₂R′ or —SCF(R′)₂ where each R′ independently represents hydrogen, halogen, C₁-C₆ alkyl, C₂-C₆ alkenyl, C₂-C₆ alkynyl or a C₃-C₆ cycloalkyl. Preferably, the aryl group is unsubstituted or substituted with one or more (preferably only one) groups chosen from C₁-C₆ alkyl (e.g. methyl), —NO₂ and —CF₃.

The term “heteroaryl” means an aromatic, mono- or polycyclic group preferably containing 5 to 20 carbon atoms and also comprising at least one heteroatom such as N, O, P, Se or S. Examples of heteroaryl are notably: pyridinyl, thiazolyl, thiophenyl, furanyl, pyrrolyl, pyrazolyl, imidazolyl, triazolyl, tetrazolyl, benzofuranyl, thianaphthalenyl, indolyl, indolinyl, quinolinyl, isoquinolinyl, benzimidazolyl, tetrahydroquinolinyl, tetrahydroisoquinolinyl, triazinyl, thianthrenyl, isobenzofuranyl, chromenyl, xanthenyl, phenoxanthinyl, isothiazolyl, isoxazolyl, pyrazinyl, pyridazinyl, indolizinyl, isoindolyl, indazolyl, purinyl, quinolizinyl, phthalazinyl, naphthyridinyl, quinoxalinyl, quinazolinyl, cinnolinyl, pteridinyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzoisoxazolyl, oxindolyl, benzoxazolinyl, benzothienyl, benzothiazolyl, isatinyl, dihydropyridyl, pyrimidinyl, s-triazinyl, oxazolyl, arylphosphine, indole, indoline, phosphindoline or thiofuranyl.

The term “halogen” means chlorine, fluorine, bromine or iodine. Preferably, a halogen is chlorine or fluorine, better still chlorine.

Said alkyl, alkenyl, alkynyl, heteroalkyl, cycloalkyl, heterocycloalkyl, aryl and heteroaryl groups as defined in the present patent application are optionally substituted. The term “optionally substituted” means unsubstituted or substituted with one or more (for example, one, two, three or four, preferably one or two, better still only one) substituents.

Examples of substituents are notably trifluoromethyl (—CF₃) and other fluoro substituents (such as —OCF₃, —SCF₃, —OCF₂R′, —OCF(R′)₂, —SCF₂R′ or —SCF(R′)₂ where each R′ independently represents hydrogen, halogen, C₁-C₆ alkyl, C₂-C₆ alkenyl, C₂-C₆ alkynyl or a C₃-C₆ cycloalkyl), nitro (—NO₂), cyano (—CN), —SO₃H, —OH, —SH, —NH₂, —COOH, halogen, C₁-C₆ alkyl, C₂-C₆ alkenyl, C₂-C₆ alkynyl, C₁-C₆ heteroalkyl, C₃-C₆ cycloalkyl, C₂-C₆ heterocycloalkyl, aryl, heteroaryl, —S(O)₂—R and —C(O)—R, in which R is independently chosen from C₁-C₆ alkyl, C₂-C₆ alkenyl, C₂-C₆ alkynyl, C₁-C₆ heteroalkyl, C₃-C₆ cycloalkyl, C₂-C₆ heterocycloalkyl, aryl, and heteroaryl.

In the present patent application, the abbreviation “Ac” means “acetyl” (i.e. —C(O)—CH₃).

The process according to the invention is a process of fluorination and/or cyclization of a substrate which is an amino alkene or alkyne. Placing the substrate in contact with a superacid reagent allows the incorporation of one or more fluorine atoms (generally one or two, and more particularly only one) onto the substrate, and/or the formation of a ring by intramolecular cyclization of the substrate. In general, the process according to the invention allows one or other among fluorination and cyclization of said amino alkene or alkyne. In a particular mode, the fluorination reaction is hydrofluorination, which consists in incorporating one or more fluorine atoms and one or more hydrogen atoms.

The process according to the invention comprises a step (a) involving providing a first phase comprising an amino alkene or alkyne, and providing a second phase comprising a superacid reagent.

In a particular embodiment, said amino alkene or alkyne is an amino alkene.

The term “amino alkene” means an organic compound containing at least one alkene function and at least one amine function. The term “alkene” means an organic compound containing at least one alkene function, i.e. at least one carbon-carbon double bond. The term “amine function” means a function:

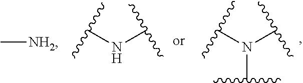

in which

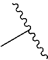

denotes a bond to the rest of the molecule. Each of the alkene and amine functions may independently be cyclic (i.e. included in a ring, such as an endocyclic alkene or a piperidine, or linked directly to the ring, such as an exocyclic alkene or a cycloalkylamine) or acyclic. The amino alkene may be aromatic or aliphatic. Preferably, the amino alkene contains from 2 to 60 carbon atoms and/or has a molecular weight of between 50 and 1000 g/mol. Needless to say, the amino alkene may also comprise groups or functions other than the alkene function(s) and the amine function(s), for example a halogen, a C₁-C₁₂ alkyl, a C₂-C₁₂ alkenyl, a C₂-C₁₂ alkynyl, a C₁-C₁₂ heteroalkyl, a C₃-C₁₂ cycloalkyl, a C₂-C₁₂ heterocycloalkyl, an aryl, heteroaryl, —CF₃ and other fluoro substituents (such as —OCF₃, —SCF₃, —OCF₂R′, —OCF(R′)₂, —SCF₂R′ or —SCF(R′)₂ where each R′ independently represents hydrogen, halogen, C₁-C₆ alkyl, C₂-C₆ alkenyl, C₂-C₆ alkynyl or a C₃-C₆ cycloalkyl), —NO₂, —CN, —SO₃H, —OH, —SH, —COOH, without this list being limiting. In particular, the amine function may be adjacent to other chemical groups or functions, such as a —C(O)— or —S(O)₂— group so that the whole constitutes an amide or a sulfonamide, respectively.

In a particular embodiment, said amino alkene is an allylamine. The term “allylamine” means a compound in which an amine function is separated from an alkene function by a carbon atom.

In a more particular embodiment, said amino alkene is a compound of formula (Ia):

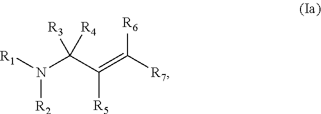

in which:
R₁, R₂, R₃, R₄, R₅, R₆ and R₇ are independently chosen from hydrogen, halogen, C₁-C₁₂ alkyl, C₂-C₁₂ alkenyl, C₂-C₁₂ alkynyl, C₁-C₁₂ heteroalkyl, C₃-C₁₂ cycloalkyl, C₂-C₁₂ heterocycloalkyl, aryl, heteroaryl, a group —S(O)₂—R₈ and a group —C(O)—R₉, where R₈ and R₉ are chosen independently from C₁-C₁₂ alkyl, C₂-C₁₂ alkenyl, C₂-C₁₂ alkynyl, C₁-C₁₂ heteroalkyl, C₃-C₁₂ cycloalkyl, C₂-C₁₂ heterocycloalkyl, aryl, and heteroaryl,
said alkyl, alkenyl, alkynyl, heteroalkyl, cycloalkyl, heterocycloalkyl, aryl and heteroaryl groups being optionally substituted, or
two groups each chosen from R₁, R₂, R₃, R₄, R₅, R₆ and R₇ (for example R₁ and R₂) may alternatively form, together with the atom(s) to which they are attached, an optionally substituted 3- to 12-membered ring.

The compound of formula (Ia) is preferably such that:
R₃ and R₄ are hydrogens, and
R₅, R₆ and R₇ are independently chosen from hydrogen and halogen (preferably at least two from among R₅, R₆ and R₇ are hydrogen). A preferred halogen is chlorine.

In a particular mode, the compound of formula (Ia) is such that R₁ and R₂ form, with the atom to which they are attached, an optionally substituted 3- to 12-membered ring, preferably a 5- to 7-membered ring, such as an optionally substituted piperidine or piperazine. In such a mode, said ring may be substituted, for example, with an optionally substituted aryl or an acetyl.

In another particular embodiment, the compound of formula (Ia) is such that:
R₁ is hydrogen, and
R₂ is an optionally substituted aryl.

In another particular embodiment, the compound of formula (Ia) is such that:
R₁ is hydrogen, and
R₂ is —S(O)₂—R₈, in which R₈ is an optionally substituted aryl.

In another particular embodiment, the compound of formula (Ia) is such that:
R₁ is a —CH₂—CH═CH₂ group, and
R₂ is —C(O)—R₉, in which R₉ is an optionally substituted aryl.

In the embodiments described above, the optionally substituted aryl group may be substituted, for example, with one or more (preferably only one) groups chosen from C₁-C₆ alkyl (e.g. methyl), —NO₂, —CF₃, and other fluoro substituents such as —OCF₃, —SCF₃, —OCF₂R′, —OCF(R′)₂, —SCF₂R′ or —SCF(R′)₂ where each R′ independently represents hydrogen, halogen, C₁-C₆ alkyl, C₂-C₆ alkenyl, C₂-C₆ alkynyl or a C₃-C₆ cycloalkyl. Preferably, the aryl group is unsubstituted or substituted with one or more (preferably only one) groups chosen from C₁-C₆ alkyl (e.g. methyl), —NO₂ and —CF₃.

In another particular embodiment, the compound of formula (Ia) is such that:
R₁ and R₂ are chosen independently from a hydrogen, a C₂-C₁₂ alkenyl, an aryl [optionally substituted with a C₁-C₆ alkyl, a nitro, or a fluoro group chosen from —CF₃, —OCF₃, —SCF₃, —OCF₂R′, —OCF(R′)₂, —SCF₂R′ and —SCF(R′)₂ where each R′ independently represents a hydrogen, a halogen, a C₁-C₆ alkyl, a C₂-C₆ alkenyl, a C₂-C₆ alkynyl or a C₃-C₆ cycloalkyl], —S(O)₂—R₈ and —C(O)—R₉, where R₈ and R₉ are independently aryl optionally substituted with C₁-C₆ alkyl, nitro or a fluoro group chosen from —CF₃, —OCF₃, —SCF₃, —OCF₂R′, —OCF(R′)₂, —SCF₂R′ and —SCF(R′)₂ where each R′ independently represents hydrogen, halogen, C₁-C₆ alkyl, C₂-C₆ alkenyl, C₂-C₆ alkynyl or a C₃-C₆ cycloalkyl,
   or alternatively R₁ and R₂, together with the atom to which they are attached, form a piperidine or piperazine, optionally substituted with an aryl or an acetyl,
R₃ and R₄ are hydrogens, and
R₅, R₆ and R₇ are independently chosen from hydrogen and halogen (preferably at least two from among R₅, R₆ and R₇ are hydrogen).

In another particular embodiment, the compound of formula (Ia) is such that:
R₁ and R₂ are independently chosen from hydrogen, C₂-C₁₂ alkenyl, aryl optionally substituted with C₁-C₆ alkyl, nitro or —CF₃, —S(O)₂—R₈ and —C(O)—R₉, in which R₈ and R₉ are independently aryl optionally substituted with C₁-C₆ alkyl, nitro or trifluoromethyl,
   or alternatively R₁ and R₂, together with the atom to which they are attached, form a piperidine or piperazine, optionally substituted with an aryl or an acetyl,
R₃ and R₄ are hydrogens, and
R₅, R₆ and R₇ are independently chosen from hydrogen and halogen (preferably at least two from among R₅, R₆ and R₇ are hydrogen).

In a preferred embodiment, said amino alkene is chosen from the following compounds:

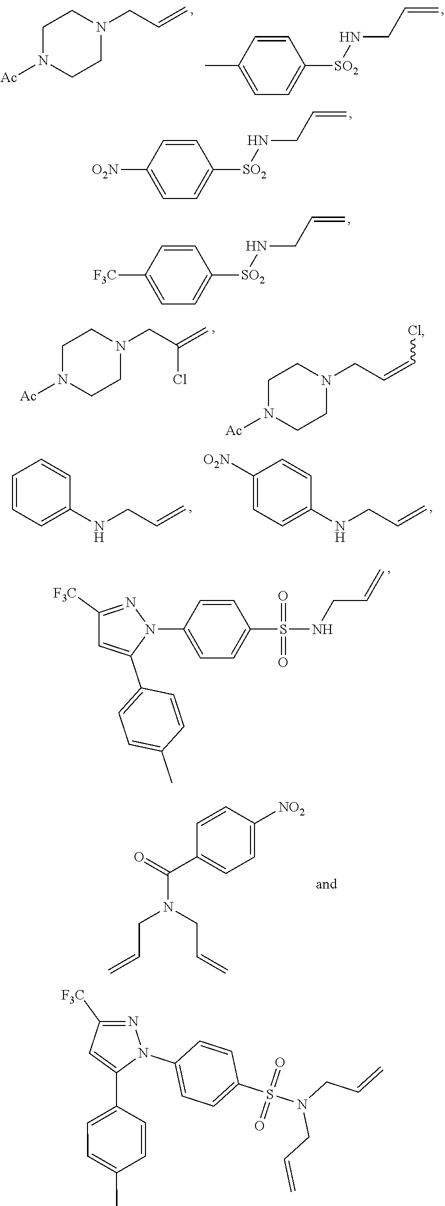

In another preferred embodiment, said amino alkene is vinorelbine, which may be represented as follows:

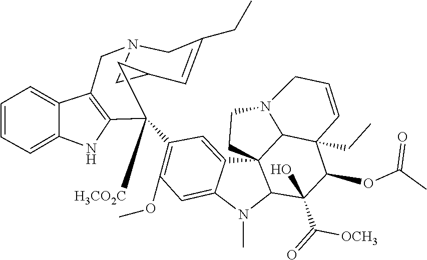

In another particular embodiment, said amino alkene or alkyne is an amino alkyne.

The term “amino alkyne” means an organic compound containing at least one alkyne function and at least one amine function. The term “alkyne” means an organic compound containing at least one alkyne function, i.e. at least one carbon-carbon triple bond. Each of the alkyne and amine functions may independently be cyclic (i.e. included in a ring, such as an endocyclic alkyne or a piperidine, or linked directly to the ring, such as a cycloalkylamine) or acyclic. The amino alkyne may be aromatic or aliphatic. Preferably, the amino alkyne contains from 2 to 60 carbon atoms and/or has a molecular weight of between 50 and 1000 g/mol. Needless to say, the amino alkyne may also comprise groups or functions other than the alkyne function(s) and the amine function(s), for example a halogen, a C₁-C₁₂ alkyl, a C₂-C₁₂ alkenyl, a C₂-C₁₂ alkynyl, a C₁-C₁₂ heteroalkyl, a C₃-C₁₂ cycloalkyl, a C₂-C₁₂ heterocycloalkyl, an aryl, a heteroaryl, —CF₃ and other fluoro groups (chosen from —OCF₃, —SCF₃, —OCF₂R′, —OCF(R′)₂, —SCF₂R′ or —SCF(R′)₂ where each R′ independently represents hydrogen, halogen, C₁-C₆ alkyl, C₂-C₆ alkenyl, C₂-C₆ alkynyl or a C₃-C₆ cycloalkyl), —NO₂, —CN, —SO₃H, —OH, —SH, —COOH, without this list being limiting. In particular, the amine function may be adjacent to other groups such as a —C(O)— or —S(O)₂— group so that the whole constitutes an amide or a sulfonamide, respectively.

In a particular embodiment, said amino alkyne is a propargyl amine. The term “propargyl amine” means a compound in which an amine function is separated from an alkyne function by a carbon atom.

In another more particular embodiment, said amino alkyne is a compound of formula (Ib):

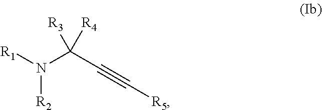

in which:
R₁, R₂, R₃, R₄ and R₅ are independently chosen from hydrogen, halogen, C₁-C₁₂ alkyl, C₂-C₁₂ alkenyl, C₂-C₁₂ alkynyl, C₁-C₁₂ heteroalkyl, C₃-C₁₂ cycloalkyl, C₂-C₁₂ heterocycloalkyl, aryl, heteroaryl, a group —S(O)₂—R₈ and a group —C(O)—R₉, where R₈ and R₉ are chosen independently from C₁-C₁₂ alkyl, C₂-C₁₂ alkenyl, C₂-C₁₂ alkynyl, C₁-C₁₂ heteroalkyl, C₃-C₁₂ cycloalkyl, C₂-C₁₂ heterocycloalkyl, aryl, and heteroaryl,
said alkyl, alkenyl, alkynyl, heteroalkyl, cycloalkyl, heterocycloalkyl, aryl and heteroaryl groups being optionally substituted, or
two groups each chosen from R₁, R₂, R₃, R₄ and R₅, (for example R₁ and R₂) may alternatively form, together with the atom(s) to which they are attached, an optionally substituted 3- to 12-membered ring.

The compound of formula (Ib) is preferably such that R₃, R₄ and R₅ are hydrogen.

In a particular mode, R₁ and R₂ form, with the atom to which they are attached, an optionally substituted 3- to 12-membered ring, preferably a 5- to 7-membered ring, such as an optionally substituted piperidine or piperazine. In such a mode, said ring may be substituted, for example, with an optionally substituted aryl or an acetyl.

According to another particular embodiment, the compound of formula (Ib) is such that:
R₁ and R₂ are chosen independently from a hydrogen, a C₂-C₁₂ alkenyl, an aryl [optionally substituted with a C₁-C₆ alkyl, a nitro, or a fluoro group chosen from —CF₃, —OCF₃, —SCF₃, —OCF₂R′, —OCF(R′)₂, —SCF₂R′ and —SCF(R′)₂ where each R′ independently represents a hydrogen, a halogen, a C₁-C₆ alkyl, a C₂-C₆ alkenyl, a C₂-C₆ alkynyl or a C₃-C₆ cycloalkyl], —S(O)₂—R₈ and —C(O)—R₉, where R₈ and R₉ are independently aryl optionally substituted with C₁-C₆ alkyl, nitro or a fluoro group chosen from —CF₃, —OCF₃, —SCF₃, —OCF₂R′, —OCF(R′)₂, —SCF₂R′ and —SCF(R′)₂ where each R′ independently represents hydrogen, halogen, C₁-C₆ alkyl, C₂-C₆ alkenyl, C₂-C₆ alkynyl or a C₃-C₆ cycloalkyl, or alternatively R₁ and R₂, together with the atom to which they are attached, form a piperidine or piperazine, optionally substituted with an aryl or an acetyl, and
R₃, R₄ and R₅ are hydrogens.

In another particular embodiment, the compound of formula (Ib) is such that:
R₁ and R₂ are independently chosen from hydrogen, C₂-C₁₂ alkenyl, aryl [optionally substituted with C₁-C₆ alkyl, nitro or —CF₃], —S(O)₂—R₈ and —C(O)—R₉, in which R₈ and R₉ are independently aryl optionally substituted with C₁-C₆ alkyl, nitro or trifluoromethyl, or alternatively R₁ and R₂, together with the atom to which they are attached, form a piperidine or piperazine, optionally substituted with an aryl or an acetyl, and
R₃, R₄ and R₅ are hydrogens.

In a preferred embodiment, said amino alkyne is chosen from the following compounds:

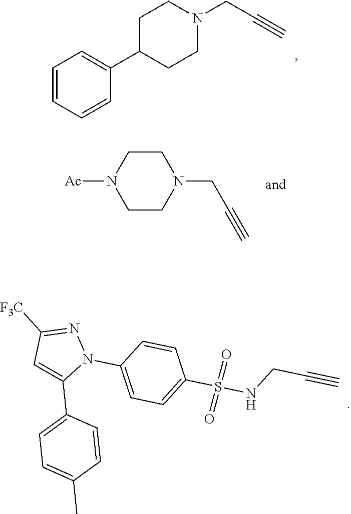

In another embodiment, said amino alkyne is chosen from the following compounds:

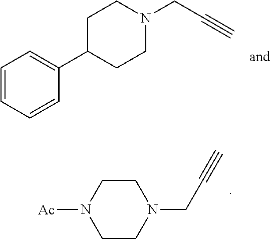

The term “superacid reagent” means a reagent with a Hammett acidity value or acidity function H₀ of less than −12, preferably less than or equal to −14. Several techniques allow this Hammett constant H₀ to be determined, notably using weak bases with spectroscopic (NMR), kinetic, thermodynamic or molecular modelling methods (Hammett L. P., Deyrup A. J., J. Am. Chem. Soc., 1932, 54 (7), 2721-2739; Superacid chemistry, Second Edition, Olah G. A., Prakash G. K. S., Molnar A., Sommer J., published by John Wiley & Sons, Inc., Hoboken, New Jersey, 2009, 1-10).

The superacid reagent may consist of an acid, which is advantageously fluorinated, or a mixture of several acids, at least one of which is advantageously fluorinated. Superacids are notably described in the following documents: Hwang, J. P.; Surya Prakash, G. K.; Olah, G. A. Tetrahedron 2000, 56 (37), 7199-7203; Culmann, J.-C.; Fauconet, M.; Jost, R.; Sommer, J. New J. Chem. 1999, 23 (8), 863-867; Esteves, P. M.; Ramírez-Solís, A.; Mota, C. J. A. J. Am. Chem. Soc. 2002, 124 (11), 2672-2677; Superacid Chemistry, Second Edition, Olah G. A., Prakash G. K. S., Molnar A., Sommer J., published by John Wiley & Sons, Inc., Hoboken, New Jersey, 2009).

The superacid reagent may notably be a Lewis superacid (such as SbF₅, AsF₅, PF₅, TaF₅), a protic or Brønsted superacid (such as HF, CF₃SO₃H, (CF₃SO₂)₂NH, HSO₃F) or a combination thereof (such as HF/SbF₅, HSO₃F/AsF₅, H₂SO₄/SO₃, HCl/AlCl₃).

Examples of superacids and their acidity constants are provided hereinbelow:
fluorosulfuric acid HSO₃F, H₀=−15.1;
trifluoromethanesulfonic acid CF₃SO₃H, H₀=−14.1;
hydrofluoric acid HF, H₀=−15.2;
the HF/SbF₅ mixture, H₀=−23/−24.

According to a particular embodiment, the superacid reagent is chosen from:
a protic superacid chosen from HF, CF₃SO₃H and HSO₃F,
a Lewis superacid of formula MF₅, where M is Sb, As, P, Ta or Nb, and
a combination of one of said protic superacids and one of said Lewis superacids.

In a particular mode, the superacid reagent is HF or HSO₃F.

In another particular mode, the superacid reagent is chosen from:
HF/MF₅, where M is Sb, As, P, Ta, or Nb; and
HSO₃F/MF₅, where M is Sb, As, P, Ta or Nb.

Preferably, the superacid reagent is HSO₃F/SbF₅ or HF/SbF₅. More preferably, the superacid reagent is HF/SbF₅.

When the superacid reagent is HF/SbF₅ or HSO₃F/SbF₅, the molar percentage of SbF₅ is advantageously less than or equal to 50%, preferably between 2% and 22%, relative to the molar amount of the HF+SbF₅ or HSO₃F+SbF₅ mixture, respectively. Needless to say, the value 0% in the range “less than or equal to 50%” is excluded.

Said first phase in step (a) is preferably liquid. The first phase may in particular consist of said amino alkene or alkyne, undiluted or in solution in a solvent. Preferably, the first phase is a solution of said amino alkene or alkyne in a solvent, such as HF, an alcohol type solvent (e.g. methanol, ethanol, or isopropanol), a fluoro alcohol type solvent (such as hexafluoropropan-2-ol (HFIP) and derivatives thereof), or a polyfluorinated or perfluorinated aromatic type solvent (such as pentafluorobenzene or 1,2,3,4-tetrafluorobenzene).

Preferably, said solvent is HF. The concentration of said amino alkene or alkyne in the solvent is advantageously between 0.02 mol/L and 2.0 mol/L, preferably between 0.5 mol/L and 1.0 mol/L.

The first phase may be stored in a storage unit, such as a tank.

In a particular mode, said first phase is maintained at a temperature of between −70° C. and 25° C., preferably between −50° C. and 5° C., better still between −50° C. and −20° C., in the storage unit.

Said second phase in step (a) is preferably liquid. The second phase advantageously consists of the undiluted superacid reagent. The second phase may be stored in a storage unit, such as a tank.

In a particular embodiment, said second phase is maintained at a temperature of between −70° C. and 25° C., preferably between −50° C. and 5° C., better still between −50° C. and −20° C., in the storage unit.

Step (b) of the process according to the invention involves placing the first and second phases in contact in a continuous-flow microreactor.

The term “continuous-flow microreactor” means a reactor of micrometric or millimetric size, allowing continuous flow of one or more fluid phases, preferably liquids. A continuous-flow microreactor typically comprises a tubular pipe, comprising an inlet and an outlet, the inside diameter of which is of micrometric or millimetric size. It is clear that, for a person skilled in the art of microfluidics, the term “microreactor” denotes a reactor whose size (more particularly, that of its inside diameter) is micrometric, but may also be millimetric insofar as this millimetric size does not affect the microfluidic properties of the microreactor, in particular with regard to the behaviour of the fluid flowing through it.

The length of the tubular pipe may be of the order of a few centimetres to several metres.

The term “micrometric size” means a size of between 1 μm and 1000 μm, preferably between 100 μm and 1000 μm, better still between 300 μm and 1000 μm (the value 1000 μm being excluded).

The term “millimetric size” means a size of between 1 mm and 10 mm, preferably between 1 mm and 5 mm.

In a particular embodiment, the tubular pipe of the continuous-flow microreactor has the following dimensions:
a length of between 20 cm and 800 cm, for instance between 30 cm and 150 cm or between 200 cm and 600 cm, and/or
an inside diameter of between 0.5 mm and 2.5 mm, preferably between 0.5 mm and 1.5 mm, and better still between 0.7 mm and 1.2 mm.

The outside diameter of the tubular pipe may be, for example, between 0.8 mm and 3.2 mm, for example between 0.8 mm and 1.0 mm, between 1.0 mm and 2.0 mm, or between 1.6 mm and 3.2 mm.

In a particular embodiment, the tubular pipe of the continuous-flow microreactor has:
an inside diameter of between 0.5 mm and 1.5 mm, and
preferably an outside diameter of between 1.0 and 2.0 mm.

The tubular pipe is generally arranged as a serpentine or coil, along all or part of its length. Other arrangements may also be envisaged, however.

Advantageously, the continuous-flow microreactor also comprises a micro-mixer. The term “micro-mixer” means a micrometric or millimetric mixer. The micro-mixer advantageously comprises two inlets and one outlet, typically giving it a T or Y shape. The inside diameter of the micro-mixer is of micrometric or millimetric size, and is advantageously between 0.5 mm and 2.5 mm, preferably between 0.5 mm and 1.5 mm, and better still between 0.7 mm and 1.2 mm. In a particular embodiment, the continuous-flow microreactor comprises:
a micro-mixer comprising two inlets and one outlet, and
a tubular pipe comprising one inlet and one outlet,
   in which the inlet of the tubular pipe is connected to the outlet of the micro-mixer. In such a mode, a means for continuously feeding said first phase is connected to the first inlet of the micro-mixer and a means for continuously feeding said second phase is connected to the second inlet of the micro-mixer.

Said continuous feed means denote any element or set of elements allowing the first and second phases to be continuously transferred into the continuous-flow microreactor, for example a pump or a syringe possibly associated with a syringe pump.

Elements are said to be “connected” when they are linked together, directly or possibly by means of a tubular pipe or other equivalent element, the characteristics of which (e.g. material, dimensions such as length and diameter) may be chosen judiciously by a person skilled in the art. For example, the micro-mixer and the tubular pipe of the continuous-flow microreactor are preferably connected directly.

The contacting step (b) may involve transferring the first phase contained in a first phase storage unit, to the continuous-flow microreactor, and in particular to the micro-mixer of the continuous-flow microreactor (even more particularly, to a first inlet of the micro-mixer) via a means for continuously feeding said first phase, and transferring the second phase contained in a second phase storage unit to the continuous-flow microreactor, and more particularly to the micro-mixer of the continuous-flow microreactor (even more particularly, to a second inlet of the micro-mixer), by means of a means for continuously feeding said second phase.

The continuous feed means for the first and second phases may be, for example, a first and a second syringes (possibly associated with a syringe pump) respectively, or a first and a second pumps respectively.

In a more particular embodiment, the first phase contained in a first phase storage unit (for example a tank) is taken up, typically by means of a first syringe or pump, and the second phase contained in a second phase storage unit (for example a tank) is taken up, typically by means of a second syringe or pump, and each of the first and second phases is injected into the continuous-flow microreactor, and more particularly into the micro-mixer of the continuous-flow microreactor (even more particularly, the first phase into a first inlet of the micro-mixer and the second phase into a second inlet of the micro-mixer). A first 3-way valve connected to the first phase storage unit, to a first syringe or pump and to the continuous-flow microreactor (in particular, to a first inlet of the micro-mixer of the microreactor), and/or a second 3-way valve connected to the second phase storage unit, to a second syringe or pump and to the continuous-flow microreactor (in particular, to a second inlet of the micro-mixer of the microreactor) may be used to facilitate the taking up and injection of the first and second phases. The inside diameter of the 3-way valves is micrometric or millimetric in size, and may be, for example, between 0.8 mm and 1.6 mm. The first and second phases may be taken up and injected using a syringe pump or a computer-controlled pump. The temperature of the syringes or pumps is generally maintained at a temperature of between −70° C. and 25° C., preferably between −50° C. and 5° C., better still between −50° C. and −20° C. The temperature of the storage units is generally maintained at a temperature of between −70° C. and 25° C., preferably between −50° C. and 5° C., better still between −50° C. and −20° C.

The flow rate of the first phase and that of the second phase in step (b) are independently between 0.1 mL/min and 3.5 mL/min, for example between 0.25 mL/min and 3.0 mL/min. The total flow rate may be between 0.5 mL/min and 4.5 mL/min, for example between 1.0 mL/min and 3.0 mL/min.

Adjusting the dimensions of the microreactor, in particular the diameter and length of a tubular pipe, and the flow rate of the first and second phases into the microreactor, allows a residence time of the first and second phases in the microreactor to be controlled and fixed. The residence time of the first and second phases placed in contact in the microreactor in step (b) is preferably between 2 seconds and 400 seconds. For example, this residence time may be between 2 and 30 seconds, between 30 and 60 seconds, or between 60 seconds and 400 seconds.

The placing in contact of the first and second phases in the continuous-flow microreactor is generally performed at a temperature (referred to as the “contacting temperature”) of between −70° C. and 25° C., preferably between −50° C. and 5° C., better still between −50° C. and −20° C. When the temperature of the first and/or second phase in step (a) is different from the contacting temperature in the microreactor in step (b), an equilibration step may be performed before placing in contact. The purpose of this equilibration is to bring to and stabilize the first and/or second phases at the contacting temperature, before placing in contact. A first equilibration tubular pipe and/or a second equilibration tubular pipe, maintained at the contacting temperature, may thus be used for this equilibration. Preferably, said first equilibration tubular pipe and/or said second equilibration tubular pipe are connected respectively:
between the first phase continuous feed means and the continuous-flow microreactor (more particularly the micro-mixer of the continuous-flow microreactor, even more particularly, a first inlet of the micro-mixer), and/or
between the second phase continuous feed means and the continuous-flow microreactor (more particularly the micro-mixer of the continuous-flow microreactor, even more particularly a second inlet of the micro-mixer).

The inside diameter of the equilibration tubular pipes is micrometric or millimetric, and may independently be between 0.5 mm and 2.5 mm, preferably between 0.5 mm and 1.5 mm, and better still between 0.7 mm and 1.2 mm. The length of the equilibration tubular pipes may independently be between 25 cm and 100 cm. The value may notably be chosen so as to allow efficient cooling at a given flow rate. The equilibration tubular pipes are generally arranged as a serpentine or coil, over all or part of their length. Other arrangements may also be envisaged, however.

The first and second phases placed in contact in the microreactor may together form one or more phases, preferably a single generally liquid phase.

Step (c) of the process according to the invention comprises the recovery (or equivalently, “collection”) of the fluorination and/or cyclization product of said amino alkene or alkyne. The product is recovered at the outlet of the continuous-flow microreactor (in particular, at the outlet of its tubular pipe), which is typically connected to a collection unit, such as a flask. The recovered product is generally recovered mixed with reaction byproducts and/or superacid reagent residues. To neutralize the superacid reagent residues, a neutralizing agent may be placed in contact with the mixture recovered in the collection unit. This neutralizing agent may be, for example, a mixture of water, a base such as sodium carbonate, sodium hydroxide, potassium hydroxide, or other bases in resin form such as Amberlyst A26 hydroxide form, and optionally one or more organic solvents such as acetone, methanol, or ammoniacal methanol. The amount of neutralizing agent to be used is adjusted according to the amount of residual superacid reagent.

Due to the corrosive nature of superacids, the elements used for performing the process of the invention, such as the continuous-flow microreactor (in particular the tubular pipe and the micromixer which it comprises), the equilibration tubular pipes, the storage units, the collection units, the syringes and/or the 3-way valves, are advantageously made of materials which are resistant to this corrosive nature. Examples of materials that may be mentioned include fluoropolymers such as polytetrafluoroethylene and poly(ethylene-co-tetrafluoroethylene) and certain metal alloys such as Hastelloy® nickel alloys. In any case, a person skilled in the art will be able to judiciously select the material suitable for each element of the assembly.

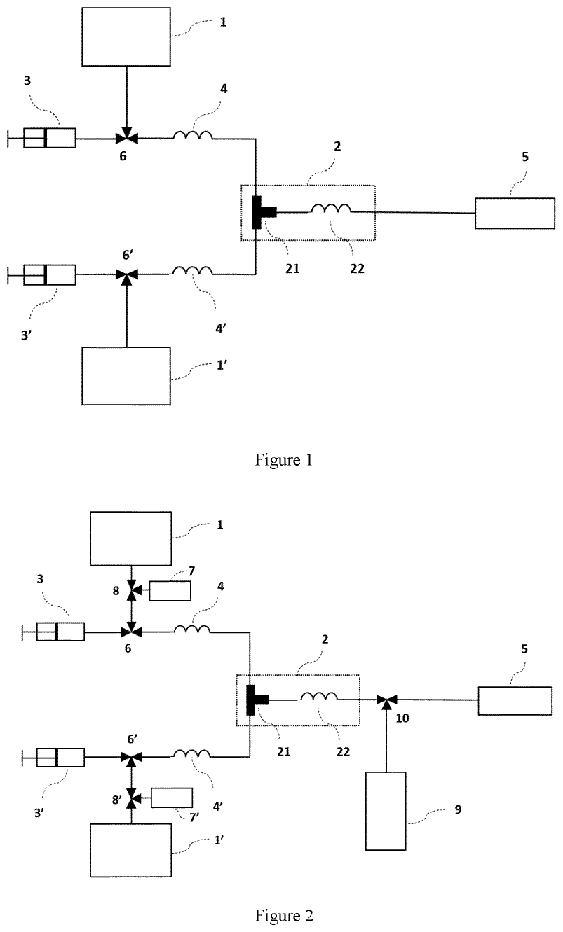

Another subject of the invention is the facility used for performing the process of the invention. FIG. 1 is a diagram illustrating the process according to one embodiment of the invention, and also the facility that may be used for performing this process.

The facility according to the invention comprises the following elements:
a storage unit (1) for a first phase comprising an amino alkene or alkyne,
a storage unit (1′) for a second phase comprising a superacid reagent,
a continuous-flow microreactor (2), preferably comprising a micro-mixer (21) and a tubular pipe (22),
a means for continuously feeding the first phase (3),
a means for continuously feeding the second phase (3′),
an equilibration tubular pipe for the first phase (4),
an equilibration tubular pipe for the second phase (4′), and
a collection unit (5).

More particularly, the facility according to the invention comprises:
a storage unit (1) for a first phase comprising an amino alkene or alkyne,
a storage unit (1′) for a second phase comprising a superacid reagent,
a first phase continuous feed means (3) connected to the first phase storage unit (1), and to a first phase equilibration tubular pipe (4),
a second phase continuous feed means (3′) connected to the second phase storage unit (1′) and to a second phase equilibration tubular pipe (4′),
a continuous-flow microreactor (2) comprising a micro-mixer (21) comprising two inlets and one outlet, and a tubular pipe (22) comprising one inlet and one outlet, in which the inlet of the tubular pipe (22) is connected to the outlet of the micro-mixer (21),
   in which the first phase equilibration tubular pipe (4) is connected to the first inlet of the micro-mixer (21) and the second phase equilibration tubular pipe (4′) is connected to the second inlet of the micro-mixer (21), and
a collection unit (5), connected to the outlet of the tubular pipe (22) of the microreactor (2).

The connection between the first-phase continuous feed means (3), the first-phase storage unit (1) and the first-phase equilibration tubular pipe (4) may be provided by means of a 3-way valve (6). The connection between the second-phase continuous feed means (3′), the second-phase storage unit (1′) and the second-phase equilibration tubular pipe (4′) may be provided by means of a 3-way valve (6′). The valves (6) and (6′) may be computer-controlled.

In a particular embodiment, the facility according to the invention also comprises one or more washing units comprising a washing solvent. FIG. 2 is a diagram illustrating the process according to one embodiment of the invention, and also the facility that may be used for performing this process, also comprising washing units. A first washing unit (7) and a second washing unit (7′) may be connected to the first phase continuous feed means (3) and the second phase continuous feed means (3′) respectively, so that the washing solvent can circulate in the continuous-flow microreactor. 3-way valves (8, 8′) may be used to alternate the injection of the first and second phases, and the injection of the washing solvent. Such 3-way valves are installed so that they connect:
on the one hand, the first phase storage unit (1), a first washing unit (7), and the first phase continuous feed means (3) (or the 3-way valve (6)), and
on the other hand, the second phase storage unit (1′), a second washing unit (7′), and the second phase continuous feed means (3′) (or the 3-way valve (6′)).

A waste collection unit (9) may be used for collecting the washing solvents after circulation through the facility. A 3-way valve (10) may be used for connecting the outlet of the tubular pipe (22) with the collection unit (5) and the waste collection unit (9).

The washing solvent may notably be water, optionally comprising a base (such as sodium carbonate, sodium hydroxide, or potassium hydroxide) and/or one or more suitable organic solvents, such as acetone. In a particular embodiment, several successive washing solvents are used, for example water, followed by an aqueous basic solution and then acetone.

The 3-way valves described in the present patent application (such as valves (6), (6′), (8), and (8′)) may, for example, be manual or automatic switching valves. The automatic switching 3-way valves may be computer controlled.

In a particular embodiment, the tubular pipe (22) of the continuous-flow microreactor (2) has the following dimensions:
a length of between 20 cm and 800 cm, for example between 30 cm and 150 cm or between 200 cm and 600 cm, and/or
an inside diameter of between 0.5 mm and 2.5 mm, preferably between 0.5 mm and 1.5 mm, and better still between 0.7 mm and 1.2 mm.

The outside diameter of the tubular pipe (22) may be, for example, between 0.8 mm and 3.2 mm, for example between 0.8 mm and 1.0 mm, between 1.0 mm and 2.0 mm, or between 1.6 mm and 3.2 mm.

In a particular embodiment, the tubular pipe (22) of the continuous-flow microreactor (2) has:
an inside diameter of between 0.5 mm and 1.5 mm, and
preferably an outside diameter of between 1.0 and 2.0 mm.

The tubular pipe (22) is generally arranged as a serpentine or coil, along all or part of its length. Other arrangements may be envisaged, however.

The micro-mixer (21) is typically T- or Y-shaped. The inside diameter of the micro-mixer (21) is of micrometric or millimetric size, and is advantageously between 0.5 mm and 2.5 mm, preferably between 0.5 mm and 1.5 mm, and better still between 0.7 mm and 1.2 mm.

Said continuous feed means (3, 3′) denote any element or set of elements allowing the first and/or second phases to be continuously transferred into the continuous-flow microreactor (2), preferably a pump or a syringe possibly associated with a syringe pump.

The inside diameter of the equilibration tubular pipes (4, 4′) is micrometric or millimetric in size, and may independently be between 0.5 mm and 2.5 mm, preferably between 0.5 mm and 1.5 mm, and better still between 0.7 mm and 1.2 mm. The length of the equilibration tubular pipes (4, 4′) may independently be between 25 cm and 100 cm. The equilibration tubular pipes (4, 4′) are generally arranged in a serpentine or coiled configuration over all or part of their length. Other arrangements may be envisaged, however.

The invention will be better understood in the light of the following examples, which are given purely by way of illustration and are not intended to limit the scope of the invention, defined by the appended claims.

### EXAMPLES

### Example 1: Fluorination and/or Cyclization of Amino Alkenes or Alkynes According to the Process of the Invention

The process for fluorinating and/or cyclizing amino alkenes or alkynes was performed via the general procedure described hereinbelow:

The facility is composed of two pumps or syringe pumps equipped with two high-pressure metal syringes each connected to an automatic valve. These syringes are connected to the fluid tanks via a metal tube. The syringes are cooled by a chamber containing dry ice, allowing the liquid in the syringe to be cooled by thermal conduction. The parts in contact with the superacid mixture have been designed and adapted to the corrosive medium by choosing a material that is compatible with this type of medium (i.e. the inside of the syringe, the inside of the 3-way valves, the metal connector tubes for transporting the fluids (inside diameter 1 mm, outside diameter 1.6 mm).

Fluids 1 (HF/SbF₅ superacid) and 2 (dissolved substrate) are respectively placed in tanks immersed in a cold bath (acetone/water, T=−35° C.) and connected to the system by tubing (inside diameter=0.8 mm, outside diameter=1.6 mm).

The 3-way valve module is a motorized ball valve, which allows the fluid passage to be opened, closed or switched. Thus, once the syringes have been filled with fluids 1 and 2, the placing in contact of the fluids may be initiated automatically via software that controls the dispensing of a small volume V of each fluid at given flow rates F1 and F2. Controlling the pumps and valves thus limits the operator's contact with the superacid during the reaction. The fluids are then conveyed to a T-shaped mixer (inside diameter 1 mm), via equilibration loops, of a length determined so as to reach the desired reaction temperature (here, L=50 cm with V=0.25 mL), which is itself connected to a tube of variable length L (inside diameter 0.8 mm, outside diameter 1.6 mm) allowing the residence time (t_{R}) of each reaction to be modulated. The collection is checked manually in flasks containing a solution to neutralize the residual acid and the product is extracted with dichloromethane or a suitable solvent, washed with water and dried over MgSO₄. The crude residue is purified by silica chromatography, then analysed by ¹H, ³C and ¹⁹F NMR and compared with the results previously obtained by the laboratory. The compounds are characterized by one or more of these techniques: ¹H, ¹³C and ¹⁹F NMR, HSQC, HMBC, HRMS.

Washing tanks (base/water then acetone/water then acetone) also connected to the manual valves allow washing to be performed at the end of the experiment.

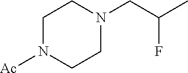

1-(4-(2-Fluoropropyl)piperazin-1-yl)ethanone 1a was obtained by following the general flow-through procedure using a tubular reactor of length L=47 cm (t_{R}=8.2 s) immersed in a bath maintained at −20° C. Flow rates of 1:1 HF/SbF₅ superacid mixture (F₁=1 mL/min) and 1-(4-allylpiperazin-1-yl)ethan-1-one substrate 1 (F₂=1 mL/min, c=0.5 mol/L) in HF were adjusted producing a final acidity of 8.3 mol % SbF₅. The reaction mixture was collected for 1 min (0.43 mmol employed, 73 mg). Purification by silica chromatography (98/2 CH₂Cl₂/MeOH) allowed 61.2 mg of product 1a (75%) to be obtained.

¹H NMR (400 MHz, CDCl₃, ppm) δ: 1.42 (dd, CH₃, J=23.8 Hz, J=6.3 Hz), 3.28 (m, CH₂), 3.98 (bs, NH). 4.89 (dm, CH, J=49.4 Hz), 6.64 (dd, 2CH, J=8.5 Hz, J=0.9 Hz), 6.74 (t, CH, J=7.3 Hz), 7.19 (dd, 2CH, J=8.4 Hz, J=7.4 Hz). ¹³C NMR (100 MHz, CDCl₃, ppm) δ: 18.8 (d, CH₃, =22 Hz), 49.6 (d, CH₂, J=21 Hz), 89.6 (d, CH, J=167 Hz), 113.1 (2CH), 118.1 (1CH), 129.4 (2CH), 147.9. ¹⁹F{¹H} NMR (CDCl₃, 376 MHz, ppm) δ: −180.0.

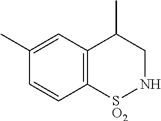

4,6-Dimethyl-3,4-dihydro-2H-benzo[e][1,2]thiazine 1,1-dioxide 2a was obtained by following the general flow-through procedure using a tubular reactor of length L=130 cm (t_{R}=52.3 s) immersed in a bath maintained at −20° C. Flow rates of HF/SbF₅ superacid mixture (F₁=0.25 mL/min) and N-allyl-4-methylbenzenesulfonamide substrate 2 in HF (c=0.5 mol/L) F₂=0.5 mL were adjusted producing a final acidity of 5.1 mol % SbF₅. The reaction mixture was collected for 2 min (0.887 mmol employed, 187.8 mg). Purification by silica chromatography (98/2 CH₂Cl₂/MeOH) allowed 171.2 mg of product 2a (76%) to be obtained.

¹H NMR (400 MHz, CDCl₃, ppm) δ: 1.34 (d, J=7.2 Hz, 3H), 2.37 (s, 3H), 2.98 (m, 1H), 3.41 (m, 1H), 3.82 (m, 1H), 4.89 (t, J=7.7 Hz, 1H), 7.09 (s, 1H), 7.14 (d, J=8.1 Hz, 1H), 7.62 (d, J=8.1 Hz, 1H). ¹³C NMR (100 MHz, CDCl₃, ppm) δ: 19.5 (CH₃), 21.6 (CH₃), 31.5 (CH), 48.2 (CH₂), 124.0 (CH), 128.2 (CH), 129.0, 134.2, 140.2, 142.7.

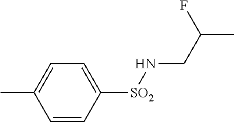

N-(2-Fluoropropyl)-4-methylbenzenesulfonamide 2b was obtained by following the general flow-through procedure using a tubular reactor of length L=78.2 cm (t_{R}=9.2 s) immersed in a bath maintained at −20° C. Flow rates of 1:1 v/v HF/SbF₅ superacid mixture (F₁=1 mL/min) and N-allyl-4-methylbenzenesulfonamide substrate 2 (F₂=2 mL/min, c=1 mol/L) in HF were adjusted producing a final acidity of 1.9 mol % SbF₅. The reaction mixture was collected for 30 sec (0.852 mmol employed, 180 mg). Purification by silica chromatography (98/2 CH₂Cl₂/MeOH) allowed 147.7 mg of product 2b (75%) to be obtained.

¹H NMR (400 MHz, CDCl₃, ppm) δ: 1.32 (dd, J=23.8, 6.3 Hz, 3H, CH₃), 2.46 (s, 3H, CH₃), 3.03 (dddd, J=18.4, 13.8, 7.6, 4.8 Hz, 1H), 3.23 (dddd, J=28.4, 13.7, 8.1, 2.9 Hz, 1H), 4.84-4.62 (dm, J_{H-F}=45 Hz, 1H), 4.84 (s, 1H), 7.35 (d, J=8 Hz, 2H), 7.77 (d, J=8.3 Hz, 2H).

¹³C NMR (100 MHz, CDCl₃, ppm) δ: 143.8 (Cq), 136.94 (Cq), 129.9 (2CH), 127.16 (2CH), 89.3 (d, J=167.6 Hz), 48.3 (CH₂, d, J=21.0 Hz), 21.7 (CH₃), 18.2 (CH₃, d, J=21.8 Hz). ¹⁹F{¹H} NMR CDCl₃, 376 MHz, ppm) δ: −180.2. HRMS (ESI): Calculated for C10H14FNO2S: 231.0729; found: 232.080241 [M+H]⁺ and 254.062169 [M+Na]⁺.

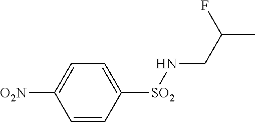 [SMILES: CC(F)CNS(=O)(=O)c1ccc([N+](=O)[O-])cc1]

N-(2-Fluoropropyl)-4-nitrobenzenesulfonamide 3b was obtained by following the general flow-through procedure using a tubular reactor of length L=78.2 cm (t_{R}=13.6 s) immersed in a bath maintained at −20° C. Flow rates of 1:1 v/v HF/SbF₅ superacid mixture (F₁=1 mL/min) and N-allyl-4-nitrobenzenesulfonamide substrate 3 (F₂=1 mL/min, c=1 mol/L) in HF were adjusted producing a final acidity of 8.4 mol % SbF₅. The reaction mixture was collected for 1 min (0.867 mmol employed, 120 mg). Purification by silica chromatography (98/2 CH₂Cl₂/MeOH) allowed 190 mg of product 3b (84%) to be obtained.

¹H NMR (400 MHz, CDCl₃, ppm) δ: 1.32 (dd, J=23.8 Hz, J=6.3 Hz, 3H), 3.10 (m, 1H), 3.26 (dm, J=28.1 Hz, 1H), 4.73 (dm, J=48.9 Hz, 1H), 5.38 (1H, m, NH), 8.07 (d, J=9.1 Hz, 2H), 8.38 (d, J=9.1 Hz, 2H). ¹³C NMR (100 MHz, CDCl₃, ppm) δ: 18.0 (d, J=22 Hz, CH₃), 48.2 (d, J=21 Hz, CH₂), 89.1 (d, J=168 Hz, CH), 124.5 (s, 2CH), 128.3 (s, 2CH), 145.8 (s), 150.1 (s). ¹⁹F{¹H} NMR (CDCl₃, 376 MHz, ppm) δ: −180.2

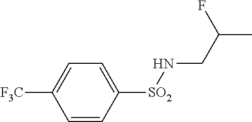 [SMILES: CC(F)CNS(=O)(=O)c1ccc(C(F)(F)F)cc1]

N-(2-Fluoropropyl)-4-(trifluoromethyl)benzenesulfonamide 4b was obtained by following the general flow-through procedure using a tubular reactor of length L=78.2 cm (t_{R}=35.7 s) immersed in a bath maintained at −20° C. Flow rates of 1:1 v/v HF/SbF₅ superacid mixture (F₁=0.25 mL/min) and N-allyl-4-trifluorobenzenesulfonamide substrate 4 (F₂=0.5 mL/min, c=0.49 mol/L) in HF were adjusted producing a final acidity of 5.2 mol % SbF₅. The reaction mixture was collected for 2 min (0.398 mmol employed, 105.6 mg). Purification by silica chromatography (98/2 CH₂Cl₂/MeOH) allowed 90.8 mg of product 4b (80%) to be obtained. No trace of the cyclization product 4a was observed under the conditions studied.

¹H NMR (400 MHz, CDCl₃, ppm) δ: 1.29 (dt, J=23.8, 6.3 Hz, 3H), 3.34-2.97 (m, 2H), 4.71 (dm, J=49 Hz, 1H), 5.24 (brs, NH), 7.78 (d, J=8.3 Hz, 2H), 8.00 (d, J=8.2 Hz, 2H). ¹⁹F{¹H} NMR (CDCl₃, 376 MHz, ppm) δ: −179.8.

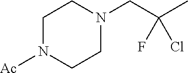

1-(4-(2-Chloro-2-fluoropropyl)piperazin-1-yl)ethan-1-one 5a was obtained by following the general flow-through procedure using a tubular reactor of length L=200 cm (t_{R}=60.3 s) immersed in a bath maintained at −20° C. Flow rates of 1:1 v/v HF/SbF₅ superacid mixture (F₁=0.5 mL/min) and 1-(4-(2-chloroallyl)piperazin-1-yl)ethan-1-one substrate 5 (F₂=0.5 m/min, c=0.5 mol/L) in HF were adjusted producing a final acidity of 6.4 mol % SbF₅. The reaction mixture was collected for 2 min (0.499 mmol employed, 111.1 mg). Purification by silica chromatography (99/1 CH₂Cl₂/MeOH) allowed 77 mg of product 5a (70%) to be obtained.

¹H NMR (400 MHz, CDCl₃, ppm) δ: 1.93 (d, J=19.4 Hz, 3H), 2.06 (s, 3H), 2.57 (m, 4H), 2.78 (dd, J=24.2 Hz, J=14.3 Hz, 1H), 2.94 (dd, J=14.3 Hz, J=14.3 Hz, 1H), 3.42 and 3.57 (2m, 4H). ¹³C NMR (100 MHz, CDCl₃, ppm) δ: 21.7 (s, CH₃), 28.4 (d, J=25 Hz, CH₃), 41.9 and 46.8 (2s, 2CH₂), 54.4 and 54.6 (s, CH₂), 67.8 (d, J=22 Hz, CH₂), 114.3 (d, J=243 Hz, 1C), 169.3 (s). ¹⁹F{¹H} NMR (CDCl₃, 376 MHz, ppm) δ: −98.9.

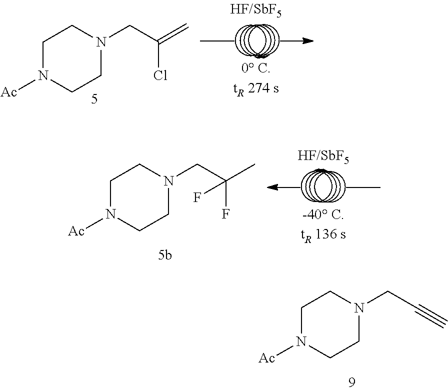

Route A (starting with compound 5): 1-(4-(2,2-Difluoropropyl)piperazin-1-yl)ethan-1-one 5b was obtained by following the general flow-through procedure using a tubular reactor of length L=600 cm (t_{R}=274 s) immersed in a bath maintained at 0° C. Flow rates of 1:1 v/v HF/SbF₅ superacid mixture (F₁=0.5 mL/min) and 1-(4-(2-chloroallyl)piperazin-1-yl)ethan-1-one substrate 5 (F₂=0.25 mL/min, c=0.499 mol/L) in HF were adjusted producing a final acidity of 12.1 mol % SbF₅. The reaction mixture was collected for 2 min (0.215 mmol employed, 44.3 mg). Purification by silica chromatography (100% CH₂Cl₂ to 98/2 CH₂Cl₂/MeOH) allowed 38 mg of product 5b (74%) to be obtained.

Route B (starting with compound 9): 1-(4-(2,2-Difluoropropyl)piperazin-1-yl)ethan-1-one 5b was obtained alternatively by following the general flow-through procedure using a tubular reactor of length L=400 cm (t_{R}=136 s) immersed in a bath maintained at −40° C. Flow rates of 3:1 v/v HF/SbF₅ superacid mixture (F₁=0.5 mL/min) and 1-(4-(prop-2-yn-1-yl)piperazin-1-yl)ethan-1-one substrate 9 (F₂=0.5 mL/min, c=0.5 mol/L) in HF were adjusted producing a final acidity of 2.65 mol % SbF₅. The reaction mixture was collected for 30 sec (0.50 mmol employed, 85.8 mg). Purification by silica chromatography (100% CH₂Cl₂ to 98/2 CH₂Cl₂/MeOH) allowed 73 mg of product 5b (71%) to be obtained.

¹H NMR (400 MHz, CDCl₃, ppm) δ: 3.66-3.59 (m, 2H), 3.47 (dd, J=12.4, 7.3 Hz, 2H), 2.70 (t, J=13.5 Hz, 2H), 2.58 (ddd, J=21.2, 13.1, 8.0 Hz, 4H), 2.09 (d, J=2.6 Hz, 3H), 1.66 (t, J=18.7 Hz, 3H). ¹⁹F{¹H} NMR (CDCl₃, 376 MHz, ppm) δ: −92.2.

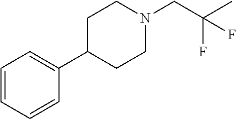 [SMILES: CC(F)(F)CN1CCC(c2ccccc2)CC1]

1-(2,2-Difluoropropyl)-4-phenylpiperidine 6a was obtained by following the general flow-through procedure using a tubular reactor of length L=200 cm (t_{R}=2.7 s) immersed in a bath maintained at −40° C. Flow rates of 3:1 v/v HF/SbF₅ superacid mixture (F₁=0.5 mL/min) and 4-phenyl-1-(prop-2-yn-1-yl)piperidine substrate 6 (F₂=1 mL/min, c=0.5 mol/L) in HF were adjusted producing a final acidity of 2.41 mol % SbF₅. The reaction mixture was collected for 30 sec (0.232 mmol employed, 46.25 mg). Purification by silica chromatography (100% CH₂Cl₂ to 98/2 CH₂Cl₂/MeOH) allowed 40.7 mg of product 6a (88%) to be obtained.

¹H NMR (400 MHz, CDCl₃, ppm) δ: 7.33-7.17 (m, 5H), 3.05 (d, J=10.8 Hz, 2H), 2.70 (t, J=13.8 Hz, 2H), 2.45 (m, 1H), 2.30 (dd, J=11.1 Hz, J=3.6 Hz, 2H), 1.79 (m, 4H), 1.66 (t, J=18.7 Hz, 3H). ¹³C NMR (100 MHz, CDCl₃) δ: 146.3, 128.4, 126.8, 126.1, 124.4 (t, J=239 Hz), 63.0 (t, J=28 Hz), 55.5, 42.2, 33.6, 22.0 (t, J=27 Hz). ¹⁹F{¹H} NMR (376 MHz, CDCl₃, ppm) δ ppm: −92.3.

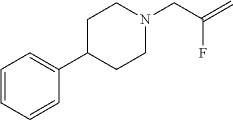 [SMILES: C=C(F)CN1CCC(c2ccccc2)CC1]

1-(2-Fluoroallyl)-4-phenylpiperidine 6b was obtained by following the general flow-through procedure using a tubular reactor of length L=30 cm (t_{R}=2.3 s) immersed in a bath maintained at −40° C. Flow rates of 3:1 v/v HF/SbF₅ superacid mixture (F₁=1 mL/min) and 4-phenyl-1-(prop-2-yn-1-yl)piperidine substrate 6 (F₂=2 mL/min, c=0.48 mol/L) in HF were adjusted producing a final acidity of 2.4 mol % SbF₅. The reaction mixture was collected for 30 sec (0.494 mmol employed, 90.2 mg). Purification by silica chromatography (100% CH₂Cl₂ to 98/2 DCM/MeOH) allowed 27.3 mg of product 6a (25%) to be obtained.

¹H NMR (400 MHz, CDCl₃, ppm) δ: 1.93-1.80 (m, 4H, 2CH₂), 2.24-2.09 (m, 2H), 2.58-2.42 (m, 1H, CH), 3.12-3.04 (m, 2H), 3.15 (d, J=16.9 Hz, 2H), 4.48 (dd, J=49.0, 2.7 Hz, 1H), 4.73 (dd, J=16.8, 2.7 Hz, 1H), 7.37-7.16 (m, 5H). ¹³C NMR (100 MHz, CDCl₃, ppm) δ: 162.8 (Cq, d, J=260.7 Hz), 146.3 (Cq), 128.44 (CH), 126.9 (CH), 126.2 (CH), 93.3 (d, J=18.7 Hz, C—F), 59.1 (d, J=27.6 Hz), 54.1 (CH₂), 42.5 (CH), 33.4 (CH₂). ¹⁹F{¹H} NMR (CDCl₃, 376 MHz, ppm) δ: −98.2. HRMS (ESI): Calculated for C14H18NF: 219.1423; found: 220.1496 [M+H]⁺.

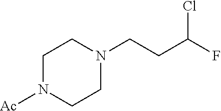 [SMILES: CC(=O)N1CCN(CCC(F)Cl)CC1]

1-(4-(3-Chloro-3-fluoropropyl)piperazin-1-yl)ethan-1-one 7a was obtained by following the general flow-through procedure using a tubular reactor of length L=400 cm (t_{R}=120.7 s) immersed in a bath maintained at −20° C. Flow rates of 1:1 v/v HF/SbF₅ superacid mixture (F₁=0.5 mL/min) and (Z/E)-1-(4-(3-chloroallyl)piperazin-1-yl)ethan-1-one substrate 7 (F₂=0.5 L/min, c=0.5 mol/L) in HF were adjusted producing a final acidity of 8.4 mol % SbF₅. The reaction mixture was collected for 1 min (0.271 mmol employed, 40.4 mg). Purification by silica chromatography (100% CH₂Cl₂ to 98/2 CH₂Cl₂/MeOH gradient) allowed 32.5 mg of product 7a (57%) to be obtained.

¹H NMR (400 MHz, CDCl₃, ppm) δ: 2.07 (s, 3H, CH₃), 2.24 (m, 2H, CH₂), 2.39 (m, 4H, 2CH₂), 3.65-3.55 (m, 2H), 3.50-3.39 (m, 2H), 2.54 (t, J=6.9 Hz, 2H), 6.29 (dt, J=50.9, 5.5 Hz, 1H, CHFCl). ¹⁹F{¹H} NMR (CDCl₃, 376 MHz, ppm) δ: −117.08.

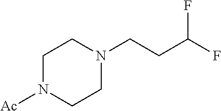 [SMILES: CC(=O)N1CCN(CCC(F)F)CC1]

1-(4-(3,3-Difluoropropyl)piperazin-1-yl)ethan-1-one 7b was obtained by following the general flow-through procedure using a tubular reactor of length L=600 cm (t_{R}=241 s) immersed in a bath maintained at 0° C. Flow rates of 1:1 v/v HF/SbF₅ superacid mixture (F₁=0.5 mL/min) and (Z/E)-1-(4-(3-chloroallyl)piperazin-1-yl)ethan-1-one substrate 7 (F₂=0.25 mL/min, c=0.5 mol/L) in HF were adjusted producing a final acidity of 12.1 mol % SbF₅. The reaction mixture was collected for 2 min (0.218 mmol employed, 44.8 mg). Purification by silica chromatography (100% CH₂Cl₂ to 98/2 CH₂Cl₂/MeOH gradient) allowed 45 mg of product 7b (85%) to be isolated.

¹H NMR (400 MHz, CDCl₃, ppm) δ: 2.05-1.92 (m, 2H, CH₂), 2.06 (s, 3H, CH₃), 2.41 (dt, J=14.7, 5.0 Hz, 4H), 2.51 (t, J=7.2 Hz, 2H), 3.44 (t, J=5.0 Hz, 2H, CH₂), 3.59 (t, J=5.1 Hz, 2H, CH₂), 5.91 (tt, J=56.7, J=4.6 Hz, 1H, CHF₂). ¹³C NMR (100 MHz, CDCl₃, ppm) δ: 168.8 (C═O), 116.2 (t, J=238.6 Hz, CH), 53.1 (CH₂), 52.5 (CH₂), 51.1 (t, J=6.3 Hz, CH₂), 46.1 (CH₂), 41.2 (CH₂), 31.5 (t, J=21.1 Hz, CH₂), 21.1 (CH₃). ¹⁹F{¹H} NMR (CDCl₃, 376 MHz, ppm) δ: −117.1.

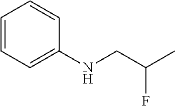

N-(2-Fluoropropyl)aniline 8a was obtained by following the general flow-through procedure using a tubular reactor of length L=50 cm (t_{R}=5.0 s) immersed in a bath maintained at −50° C. Flow rates of 3:1 v/v HF/SbF₅ superacid mixture (F₁=1.5 mL/min) and N-allylaniline substrate 8 (F₂=1.5 mL/min, c=0.57 mol/L) in HF were adjusted producing a final acidity of 3.74 mol % SbF₅. The reaction mixture was collected for 30 sec (0.429 mmol employed, 53.6 mg). Purification by silica chromatography (100% CH₂Cl₂ to 98/2 CH₂Cl₂/MeOH gradient) allowed 43 mg of product 8a (66%) to be isolated.

¹H NMR (400 MHz, CDCl₃, ppm) δ: 1.43 (dd, J=23.8, 6.3 Hz, 3H, CH₃), 3.44-3.17 (m, 2H), 5.01-4.77 (dm, J=49.56 Hz, 1H, CH—F), 6.64 (m, 2H, CHₐᵣ), 6.75 (tt, J=7.4, 1.0 Hz, 1H), 7.24-7.12 (m, 2H, CH). ¹⁹F{¹H} NMR (CDCl₃, 376 MHz, ppm) δ: −179.9.

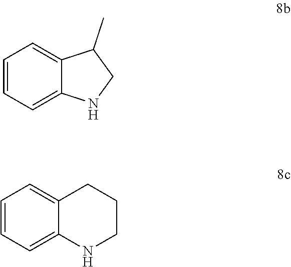

3-Methylindoline 8b and 1,2,3,4-tetrahydroquinoline 8c were obtained by following the general flow-through procedure using a tubular reactor of length L=200 cm (t_{R}=40 s) immersed in a bath maintained at 0° C. Flow rates of 1:1 v/v HF/SbF₅ superacid mixture (F₁=0.5 mL/min) and N-allylaniline substrate 8 (F₂=1.0 mL/min, c=0.74 mol/L) in HF were adjusted producing a final acidity of 5.2 mol % SbF₅. The reaction mixture was collected for 30 sec (0.429 mmol employed, 53.6 mg). Purification by silica chromatography (CH₂Cl₂/MeOH gradient) allowed 31.3 mg of product 8b (37%) and 35.9 mg of product 8c (42%) to be isolated. 3-methylindoline 8b ¹H NMR (400 MHz, CDCl₃, ppm) δ: 1.33 (d, J=6.8 Hz, 1H), 3.12 (t, J=8.6 Hz, 1H), 3.45-3.27 (m, 1H), 3.71 (t, J=8.6 Hz, 1H), 6.66 (d, J=7.7 Hz, 1H), 6.75 (td, J=7.4, 0.9 Hz, 1H), 7.04 (ddd, J=8.7, 2.0, 1.0 Hz, 1H), 7.12-7.08 (m, 1H), 1,2,3,4-tetrahydroquinoline 8c ¹H NMR (400 MHz, CDCl₃, ppm) δ: 1.95 (dtd, J=8.8, 6.4, 4.2 Hz, 1H), 2.77 (t, J=6.4 Hz, 1H), 3.33-3.28 (m, 1H), 6.48 (d, J=7.9 Hz, 1H), 3.78 (s, 1H), 6.61 (td, J=7.4, 1.1 Hz, 1H), 7.00-6.89 (m, 1H).

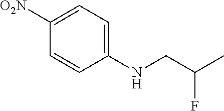

N-(2-Fluoropropyl)-4-nitroaniline 12b was obtained by following the general flow-through procedure using a tubular reactor of length L=200 cm (t_{R}=40 s) immersed in a bath maintained at 0° C. Flow rates of 1:1 v/v HF/SbF₅ superacid mixture (F₁=0.5 mL/min) and N-allyl-4-nitroaniline substrate (F₂=1 mL/min, c=0.3 mol/L) in HF were adjusted producing a final acidity of 5.1 mol % SbF₅. The reaction mixture was collected for 1 min 21 sec (0.448 mmol employed, 79.8 mg). Purification by silica chromatography (CH₂Cl₂/MeOH gradient) allowed 65 mg of product 12b (73%) to be isolated.

With a tubular reactor L=600 cm (t_{R}=241 s), F₁=0.25 mL/min and F₂=0.5 mL/min a yield of 92% of product 12b was obtained.

¹H NMR (400 MHz, CDCl₃, ppm) δ: 1.44 (dd, J=23.8, 6.3 Hz, 3H, CH₃), 3.40 (m, 2H, CH₂), 4.79 (bs, 1H, NH), 4.87 (dm, J=49.1, ³J_{H-H}=6.3 Hz, CFH), 6.57 (d, J=9.3 Hz, 2-H, CH), 8.09 (d, J=9.3 Hz, 2H, CH). ¹⁹F{¹H} NMR (CDCl₃, 376 MHz, ppm) δ: −179.9

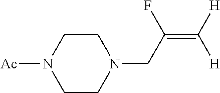 [SMILES: C=C(F)CN1CCN(C(C)=O)CC1]

1-(4-(2-Fluoroallyl)piperazin-1-yl)ethan-1-one 9b was obtained by following the general flow-through procedure using a tubular reactor of length L=30 cm (t_{R}=2 s) immersed in a bath maintained at −40° C. Flow rates of 5:1 v/v HF/SbF₅ superacid mixture (F₁=1.5 mL/min) and 1-(4-(prop-2-yn-1-yl)piperazin-1-yl)ethan-1-one substrate 9 (F₂=3 mL/min, c=0.5 mol/L) in HF were adjusted producing a final acidity of 1.57 mol % SbF₅. The reaction mixture was collected for 30 sec (0.748 mmol employed, 154.3 mg). Purification by silica chromatography (100% CH₂Cl₂ to 98/2 CH₂Cl₂/MeOH) allowed 97.9 mg of product 9b (70%) to be isolated.

¹H NMR (400 MHz, CDCl₃, ppm) δ: 2.09 (s, 3H, CH₃), 2.50 (m, 4H, 2CH₂), 3.11 (dd, J=16.5 Hz, J=2.7 Hz, CH₂), 3.51 (m, 2H, CH₂), 3.65 (m, 2H, CH₂), 4.47 (dt, J=48.8 Hz, J=2.8 Hz, CH), 4.74 (dt, J=16.6 Hz, J=2.8 Hz, CH). ¹³C NMR (100 MHz, CDCl₃, ppm) δ: 41.2 (CH₂), 46.1 (CH₂), 58.4 (d, J=Hz, CH₂), 93.8 (CH₂), ¹⁹F{¹H} NMR (CDCl₃, 376 MHz, ppm) δ: −98.7. HRMS (ESI): Calculated for C₉H₁₅FN₂O: 186.1168; found: 187.1241 [M+H]⁺.

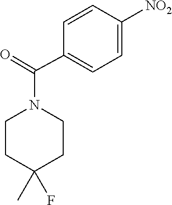 [SMILES: CC1(F)CCN(C(=O)c2ccc([N+](=O)[O-])cc2)CC1]

(4-Fluoro-4-methylpiperidin-1-yl)(4-nitrophenyl)methanone 10a was obtained by following the general flow-through procedure using a tubular reactor of length L=600 cm (t_{R}=104 s) immersed in a bath maintained at 0° C. Flow rates of 1:1 v/v HF/SbF₅ superacid mixture (F1=1 mL/min) and N,N-diallyl-4-nitrobenzamide substrate 10 (F₂=1 mL/min, c=0.23 mol/L) in HF were adjusted producing a final acidity of 8.4 mol % SbF₅. The reaction mixture was collected for 1 min 53 sec (0.353 mmol employed, 75.7 mg). Purification by silica chromatography (70/30 petroleum ether/EtOAc) allowed 43.1 mg of product 10a (46%) to be isolated.

¹H NMR (400 MHz, CDCl₃, ppm) δ: 1.41 (d, J=21.4 Hz, 3H, CH₃), 1.71 (m, 4H, 2CH₂), 3.17 (m, 1H), 4.53 (m, 1H), 3.41 (m, 2H, CH₂), 7.56 (d, J=8.8 Hz, 2H, 2CH), 8.27 (d, J=8.8 Hz, 2H, 2CH). ¹⁹F{¹H} NMR (CDCl₃, 376 MHz, ppm) δ: −151.7.

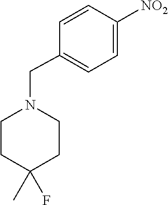 [SMILES: CC1(F)CCN(Cc2ccc([N+](=O)[O-])cc2)CC1]

4-Fluoro-4-methyl-1-(4-nitrobenzyl)piperidine 16a was obtained by following the general flow-through procedure using a tubular reactor of length L=600 cm (t_{R}=104 s) immersed in a bath maintained at 0° C. Flow rates of 1:1 v/v HF/SbF₅ superacid mixture (F₁=1 mL/min) and N-allyl-N-(4-nitrobenzyl)prop-2-en-1-amine substrate 16 (F₂=1 mL/min, c=0.34 mol/L) in HF were adjusted producing a final acidity of 8.4 mol % SbF₅. The reaction mixture was collected for 1 min (0.341 mmol employed, 88.4 mg). Purification by silica chromatography (99/1 CH₂Cl₂/MeOH) allowed 37 mg of product 16a (47%) to be isolated.

¹H NMR (400 MHz, CDCl₃, ppm) δ: 1.35 (d, J=21.6 Hz, CH₃), 1.66 and 1.85 (m, 2CH), 2.26 (m, 2CH), 2.60 (m, 2CH), 3.62 (s, CH₂), 7.52 (d, J=8.8 Hz, 2CHₐᵣ), 8.17 (d, J=8.8, 2CHₐᵣ). ¹³C NMR (100 MHz, CDCl₃, ppm) δ: 26.9 (d, J=24 Hz, CH₃), 36.6 (d, J=22 Hz, 2CH₂), 49.5 (d, J=1.1 Hz, 2CH₂), 62.1 (CH₂), 91.9 (d, J=167 Hz, C—F), 123.5 (2CH), 129.4 (2CH), 146.8 (Cq), 147.1 (Cq). ¹⁹F{¹H} NMR (CDCl₃, 376 MHz, ppm) δ: −151.55. HRMS (ESI): Calculated for C₁₃H₁₈FN₂O₂: 252.1274; found: 253.1346 [M+H]⁺.

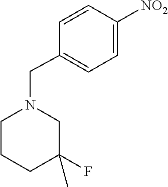 [SMILES: CC1(F)CCCN(Cc2ccc([N+](=O)[O-])cc2)C1]

3-Fluoro-3-methyl-1-(4-nitrobenzyl)piperidine 16b was obtained by following the general flow-through procedure using a tubular reactor of length L=600 cm (t_{R}=104 s) immersed in a bath maintained at −40° C. Flow rates of 1:1 v/v HF/SbF₅ superacid mixture (F₁=1 mL/min) and N-allyl-N-(4-nitrobenzyl)prop-2-en-1-amine substrate 16 (F₂=1 mL/min, c=0.34 mol/L) in HF were adjusted producing a final acidity of 8.4 mol % SbF₅. The reaction mixture was collected for 1 min (0.341 mmol employed, 88.4 mg). Purification by silica chromatography (99/1 CH₂Cl₂/MeOH) allowed 12 mg of 3-fluoro-3-methyl-1-(4-nitrobenzyl)piperidine 16b (35%) to be isolated.

¹H NMR (400 MHz, CDCl₃, ppm) δ: 1.34 (d, J=21.6 Hz, CH₃), 1.53 (m, 2CH), 1.83 (m, 2CH), 2.22 (m, 2CH), 2.55 (m, 2CH), 3.61 (s, CH₂), 7.51 (d, J=8.8 Hz, 2CHₐᵣ), 8.16 (d, J=8.7 Hz, 2CHₐᵣ). ¹³C NMR (100 MHz, CDCl₃, ppm) δ: 22.0 (d, J=4 Hz, CH₂), 25.0 (d, J=24 Hz, CH₃), 35.0 (d, J=22.1 Hz, CH₂), 53.1 (CH₂), 61.8 (CH₂), 62.1 (d, J=23 Hz, CH₂), 92.2 (d, J=170 Hz, Cq), 123.5 (2CH), 129.3 (CH), 146.4 (Cq), 147.1 (Cq). ¹⁹F{¹H} NMR (CDCl₃, 376 MHz, ppm) δ: −146.8. HRMS (ESI): Calculated for C₁₃H₁₈FN₂O₂: 252.1274; found: 253.1346 [M+H]⁺.

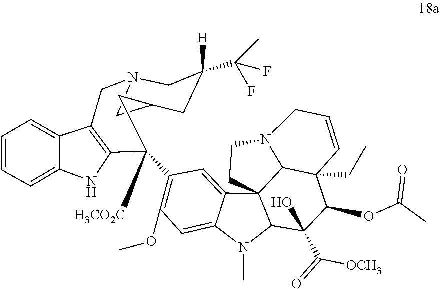

Vinflunine 18a was obtained following the general procedure using a tubular reactor of length L=600 cm (t_{R}=603 s) immersed in a bath maintained at −40° C. Flow rates F₁=0.2 or 0.3 mL/min of 2:1 v/v HF/SbF₅ mixture and F₂=0.2 or 0.3 mL/min of vinorelbine tartrate 18 dissolved in anhydrous chloroform (c=0.03 mol/L) were applied. The reaction mixture was collected for 10 min, allowing 23 mg of crude residue to be collected. NMR and HPLC analysis of the crude mixture allowed vinflunine to be identified in the predominant amount with yields of about 3-15% (NMR yields) relative to the standard reference. Purification of the crude mixture using a C18 reverse phase (ACN/TFA aq. 0.2% gradient peak of interest isolated at 30% ACN) allowed the isolation of the enriched fractions (HPLC).

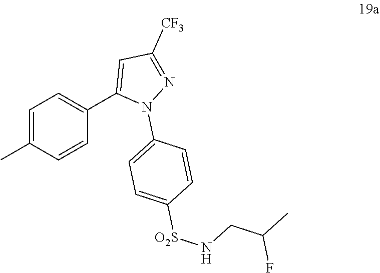 [SMILES: Cc1ccc(-c2cc(C(F)(F)F)nn2-c2ccc(S(=O)(=O)NCC(C)F)cc2)cc1]

N-(2-Fluoropropyl)-4-(5-(p-tolyl)-3-(trifluoromethyl)-1H-pyrazol-1-yl)benzenesulfonamide 19a was obtained following the general flow-through procedure using a tubular reactor of length L=78 cm (t_{R}=7.8 s) immersed in a dry-ice bath. Flow rates of HF/SbF₅ superacid mixture and N-allyl-4-(5-(p-tolyl)-3-(trifluoromethyl)-1H-pyrazol-1-yl)benzenesulfonamide 19 (c=0.16 mol/L) were adjusted. The reaction mixture was collected for 30 sec (0.156 mmol employed, 65.6 mg). Purification by silica chromatography (PE/EtOAc gradient from 5% to 30% of EtOAc) allowed 50.2 mg of product 19a (72%) to be obtained.

¹H NMR (400 MHz, CDCl₃, ppm) δ: 1.29 (dd, J=23.8, 6.3 Hz, 3H), 2.38 (s, 3H), 3.02 (m, 1H), 3.21 (m, 1H), 4.69 (dm, J=49.2 Hz, 1H), 5.07 (bm, 1H), 6.75 (s, 1H), 7.10 (d, J=8.2 Hz, 1H), 7.17 (d, J=7.9 Hz, 1H), 7.54-7.43 (m, 1H), 7.91-7.79 (m, 1H). ¹³C NMR (100 MHz, CDCl₃, ppm) δ: 145.4, 144.2 (q, J=38.5 Hz, CF₃), 142.7, 139.9, 139.4, 129.87 (s), 128.8 (s), 128.1 (s), 125.7, 121.1 (q, J=269.2 Hz, CF), 106.4, 89.8 (s), 88.5, 48.2 (d, J=21.1 Hz, CF), 21.4, 18.2, 18.1. ¹⁹F NMR (CDCl₃, 376 MHz, ppm) δ: −62.42 (CF₃), −179.9 (CF). HRMS (ESI): Calculated for C₂₀H₁₉F₄N₃O₂S 441.1134; found C₂₀H₁₉F4N₃O₂S 442.1206 [M+H]⁺.

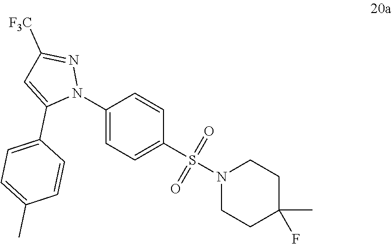 [SMILES: Cc1ccc(-c2cc(C(F)(F)F)nn2-c2ccc(S(=O)(=O)N3CCC(C)(F)CC3)cc2)cc1]

4-Fluoro-4-methyl-1-((4-(5-(p-tolyl)-3-(trifluoromethyl)-1H-pyrazol-1-yl)phenyl)sulfonyl)piperidine 20a was obtained following the general flow-through procedure using a tubular reactor of length L=400 cm (t_{R}=4.1 s) immersed in an acetone/dry ice bath. Flow rates of HF/SbF₅ superacid mixture and N-diallyl-4-(5-(p-tolyl)-3-(trifluoromethyl)-1H-pyrazol-1-yl)benzenesulfonamide 20 (c=0.11 mol/L) were adjusted. The reaction mixture was collected for 2 min (0.076 mmol employed, 37 mg). Purification by silica chromatography (PE/EtOAc gradient from 5% to 30% of EtOAc) allowed 7.2 mg of product 20a (20%) to be obtained.

¹H NMR (400 MHz, CDCl₃, ppm) δ: 7.77 (d, J=8.7 Hz, 1H), 7.48 (d, J=8.7 Hz, 1H), 7.17 (d, J=7.9 Hz, 1H), 7.09 (d, J=8.2 Hz, 1H), 6.75 (s, 1H), 3.58-3.28 (m, 1H), 2.88-2.54 (m, 1H), 2.76-2.64 (m, 1H), 2.38 (s, 1H), 1.89-1.77 (m, 1H), 1.65-1.54 (m, 1H), 1.36 (d, J=21.0 Hz, 1H). ¹³C NMR (100 MHz, CDCl₃, ppm) δ: 145.4, 144.2 (q, J=38.5 Hz), 142.6, 139.9, 136.7, 129.8, 128.8, 128.7, 125.71, 122.2, 120.1, 106.3, 90.5 (d, J=175.4 Hz), 53.9 (d, J=25.7 Hz), 45.5, 34.5 (d, J=22.6 Hz), 24.5 (d, J=23.4 Hz), 21.4, 21.11 (d, J=3.6 Hz). ¹⁹F{¹H} NMR (CDCl₃, 376 MHz, ppm) δ: −62.44, −150.01. HRMS (ESI): Calculated for C₂₃H₂₄F₄N₃O₂S [M+H]482.151987; found 482.154140.

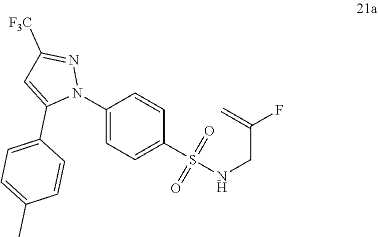 [SMILES: C=C(F)CNS(=O)(=O)c1ccc(-n2nc(C(F)(F)F)cc2-c2ccc(C)cc2)cc1]

N-(2-Fluoroallyl)-4-(5-(p-tolyl)-3-(trifluoromethyl)-1H-pyrazol-1-yl)benzenesulfonamide 21a was obtained following the general flow-through procedure using a tubular reactor of length L=25 cm (t_{R}=4.2 s) immersed in an acetone/dry ice bath. Flow rates of HF/SbF₅ superacid mixture and N-(prop-2-yn-1-yl)-4-(5-(p-tolyl)-3-(trifluoromethyl)-1H-pyrazol-1-yl)benzenesulfonamide 21 (c=0.11 mol/L) were adjusted. The reaction mixture was collected for 30 sec (0.07 mmol employed, 31 mg). Purification by silica chromatography (PE/EtOAc—0% to 10% of EtOAc) allowed the fluorovinyl compound 21a to be obtained in a yield of 29%.

¹H NMR (400 MHz, CDCl₃, ppm) δ: 7.85 (d, J=8.7 Hz, 1H), 7.47 (d, J=8.7 Hz, 1H), 7.17 (d, J=8.0 Hz, 1H), 7.10 (d, J=8.1 Hz, 1H), 6.74 (s, 1H), 4.77 (t, J=6.4 Hz, 1H), 4.61 (dd, J=16.2, 3.5 Hz, 1H), 4.44 (dd, J=47.9, 3.5 Hz, 1H), 3.76 (dd, J=13.1, 6.3 Hz, 1H), 2.38 (s, 2H). ¹³C NMR (100 MHz, CDCl₃, ppm) δ: 161.12, 159.07, 145.26, 144.13 (q, J=39.0 Hz), 142.69, 139.83, 139.36, 129.77, 128.73, 128.14, 125.67, 125.64, 125.55, 106.38, 93.36 (d, J=17.4 Hz), 43.45 (d, J=32.6 Hz), 29.73, 21.37. ¹⁹F{¹H} NMR (CDCl₃, 376 MHz, ppm) δ: −62.44 (s), −104.12-−104.45 (m). HRMS (ESI): Calculated for C₂₀H₁₈F₄N₃O₂S [M+H]: 440.105037; found: 440.106449.

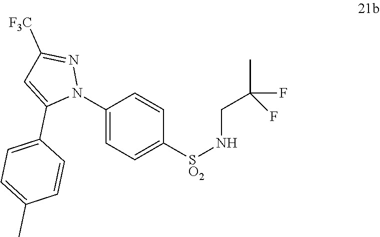 [SMILES: Cc1ccc(-c2cc(C(F)(F)F)nn2-c2ccc(S(=O)(=O)NCC(C)(F)F)cc2)cc1]

N-(2,2-Difluoropropyl)-4-(5-(p-tolyl)-3-(trifluoromethyl)-1H-pyrazol-1-yl)benzenesulfonamide 21b was obtained following the general flow-through procedure using a tubular reactor of length L=39 cm (t_{R}=2 s) at low temperature. Flow rates of HF/SbF₅ superacid mixture and N-(prop-2-yn-1-yl)-4-(5-(p-tolyl)-3-(trifluoromethyl)-1H-pyrazol-1-yl)benzenesulfonamide 21 were adjusted. The reaction mixture was collected for 15 sec (0.118 mmol employed, 49.5 mg). Purification by silica chromatography (PE/EtOAc gradient) allowed 47.2 mg of product 21b (86%) to be obtained.

¹H NMR (500 MHz, CDCl₃, ppm) δ: 1.65 (t, J=18.6, 3H), 2.40 (s, 3H), 3.35 (td, J=13.0, 6.8 Hz, 2H), 5.08 (bs, 1H), 6.77 (s, 1H), 7.12 (d, J=8.1 Hz, 2H), 7.20 (d, J=7.9 Hz, 2H), 7.51 (m, 2H), 7.86 (m, 2H). ¹³C NMR (126 MHz, CDCl₃, ppm) δ: 145.3, 144.1 (q, J=38.6 Hz, CF₃), 142.7, 139.8, 139.2, 129.7, 128.7, 127.9, 125.6, 125.6, 123.3, 121.4, 121.0 (q, J=269.2 Hz, CF₂), 119.5, 106.4, 106.34 (s, J=1.4 Hz), 48.15 (t, J=30.9 Hz), 29.7, 21.4, 21.3, 21.2, 21.0. ¹⁹F NMR (CDCl₃, 376 MHz, ppm) δ: −62.4 (s), −96.6 (m). HRMS (ESI): Calculated for C₂₀CH₁₈F₅N₃O₂S 459.1040; found C₂₀H₁₉F5N₃O₂S 460.1112 [M+H]⁺.

### Example 2: Comparative Study

### 1) Productivity

The productivity of each reaction was calculated, giving access to the amount of product formed in a given time. The productivity is given relative to the volume of the reactor, so that reactors of different size or construction can be compared with each other. Similarly, the productivity may be calculated for a static reaction volume and compared with the reactor volume over a given time in flow chemistry. This is known as the Space-Time Yield (STY) in kg·m⁻³h⁻¹.

$$STY = \frac{m of product ( in g )}{volume of reactor ( mL ) \times reaction time ( min )}$$

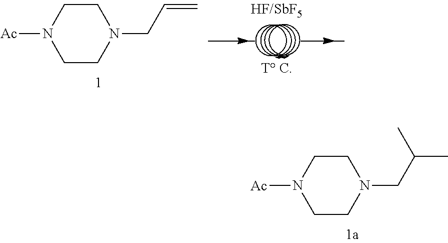

For example, in the case of compound 1, it may be noted that the mass productivity of the fluorination reaction increases by a factor of 34 on going from the static method to the process described in the invention, which is a flow process.

TABLE 1

| | | Invention |
|---|---|---|
| | Static | (flow) |
| Yield (%) | 69 | 75 |
| Reaction time t_{R} in min | 10 | 0.14 |
| Reaction volume (mL) | 6 | 0.24 |
| m product (g) | 0.13 | 0.062 |
| n product (mmol) | 0.689 | 0.325 |
| Productivity (mmol/min) | 0.0698 | 2.32 |
| Productivity (g/h) | 0.78 | 26.6 |
| STY (kg · m⁻³ · h⁻¹) | 130 | 110714 |

The productivities of the static process and the process of the invention for each reaction product were calculated from the data summarized in the table hereinbelow (Table 2). In general, the process according to the invention is much more efficient, notably for reactions with a very short residence time (t_{R}<1 min), allowing the mass productivity to be multiplied by a factor of up to 34 relative to the static process.

The acidity of the medium is an important parameter, as mentioned previously. Exploitation of the variation in acidity was also studied in the flow process and, for several syntheses, the amount of SbF₅ was considerably reduced, while at the same time increasing the efficacy of the reaction and obtaining higher productivity relative to the static process.

TABLE 2

| | | n | V | | | | | | |
|---|---|---|---|---|---|---|---|---|---|
| | | Prod. | reactor | t_{R} | mol % | Yield | W mass | STY | |
| Product | Process | mmol | ml | min | SbF₅ | % | g/h | g · m⁻³ · h⁻¹ | ref |
| 1a | Invention | 0.325 | 0.24 | 0.14 | 8.4 | 75 | 26.6 | 110714 | — |
| | Static | 0.689 | 6.0 | 10 | 3.8 | 69 | 0.78 | 130 | i |
| 2a | Invention | 0.810 | 0.39 | 0.87 | 5.2 | 76 | 11.8 | 30274 | — |
| | Static | 1.280 | 6.0 | 10 | 13.6 | 64 | 0.81 | 270 | i |
| 2b | Invention | 0.639 | 0.39 | 0.15 | 2.41 | 75 | 59.1 | 151487 | — |
| | Static | — | — | — | — | — | — | — | — |
| 3b | Invention | 0.728 | 0.39 | 0.23 | 8.4 | 84 | 49.8 | 127759 | — |
| | Static | 0.923 | 6.0 | 10 | 13.6 | 74 | 1.45 | 242 | i |
| 4b | Invention | 0.318 | 0.39 | 0.60 | 5.2 | 80 | 9.16 | 23478 | — |
| | Static | 0.830 | 3.0 | 10 | 3.8 | 84 | 1.43 | 476 | ii |
| 5a | Invention | 0.499 | 1.0 | 1 | 6.4 | 70 | 4.60 | 11787 | — |
| | Static | 0.740 | 3.0 | 10 | 3.8 | 74 | 0.98 | 130 | iii |
| 5b | Invention | 0.160 | 1.0 | 1 | 3.7 | 64 | 1.97 | 1968 | — |
| starting | Invention | 0.350 | 2.0 | 4.02 | 3.7 | 71 | 1.09 | 545 | — |
| from 9 | Static | — | — | — | — | — | — | — | — |
| 6a | Invention | 0.170 | 0.15 | 0.045 | 5.1 | 92 | 54.0 | 360000 | — |
| | Static | 1.316 | 7.5 | 10 | 12.6 | 87 | 1.89 | 252 | iv |
| 6b | Invention | 0.124 | 0.15 | 0.058 | 2.1 | 27 | 28.2 | 188276 | — |
| | Static | — | — | — | — | — | — | — | — |
| 8a | Invention | 0.324 | 0.25 | 0.10 | 3.7 | 66 | 26.7 | 106639 | |
| | Static | 0.571 | 4.0 | 10.00 | 3.8 | 50 | 0.46 | 114 | v |
| 8b | Invention | 0.235 | 1.0 | 0.77 | 3.7 | 42 | 2.4 | 2439 | — |
| | Static | 0.367 | 4.0 | 10.0 | 21.6 | 39 | 0.30 | 74 | v |
| 8c | Invention | 0.269 | 1.0 | 0.77 | 3.7 | 37 | 2.8 | 2797 | — |
| | Static | 0.210 | 4.0 | 10.0 | 21.6 | 22 | 0.17 | 42 | v |
| 9b | Invention | 0.525 | 0.15 | 0.033 | 1.6 | 70 | 178.0 | 1186667 | — |
| | Static | — | — | — | — | — | — | — | — |
| 19a | Invention | 0.113 | 0.39 | 0.13 | 3.7 | 72 | 23.2 | 59374 | — |
| | Static | — | — | — | — | — | — | — | — |
| 20a | Invention | 0.0264 | 2.01 | 4.0 | 1.0 | 35 | 0.19 | 59374 | — |
| | Static | — | — | — | — | — | — | — | — |
| 21a | Invention | 0.020 | 0.13 | 0.07 | 0.94 | 29 | 7.63 | 58681 | — |
| | Static | — | — | — | — | — | — | — | — |
| 21b | Invention | 0.103 | 0.15 | 0.033 | 2.4 | 87 | 85.6 | 570909 | — |
| | Static | 0.088 | 4.0 | 10.0 | 12.0 | 73 | 0.24 | 60 | — |

i Sébastien Thibaudeau, Agnès Martin-Mingot, Marie-Paule Jouannetaud, Omar Karam, Fabien Zunino Chem. Commun 2007, 3198-3200.
ii Fei Liu, Agnès Martin-Mingot, Marie-Paule Jouannetaud, Fabien Zunino, Sébastien Thibaudeau Org. Lett. 2010, 12(4), 868-871.
iii Fei Liu, Agnès Martin-Mingot, Marie-Paule Jouannetaud, Christian Bachmann, Gilles Frapper, Fabien Zunino, Sébastien Thibaudeau J. Org. Chem. 2011, 76, 1460-1463.
iv Cantet, Anne-Céline; Carreyre, Helene; Gesson, Jean-Pierre; Jouannetaud, Marie-Paule; Renoux, Brigitte J. Org. Chem. 2008, 73(7), 2875-2878.
v a) Guillaume Compain, Agnès Martin-Mingot, Gilles Frapper, Christian Bachmann, Marie-Paule Jouannetaud, Sébastien Thibaudeau Chem. Commun., 2012, 48, 5877-5879. b) Guillaume Compain, Céline Bonneau, Agnès Martin-Mingot, Sébastien Thibaudeau J. Org. Chem. 2013, 78, 4463-4472.

### 2) Selectivity

The application of a superacid reagent to compound 2 in static mode systematically produces only compound 2a, while the process of the invention allows compound 2a or 2b to be obtained selectively by adjusting the residence time and the concentration of superacid reagent.

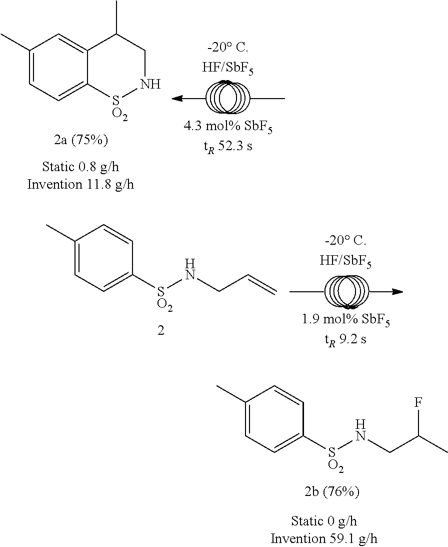

In addition, applying a superacid reagent to compound 9 in static mode systematically produces only the difluoro compound 5b, whereas the process of the invention allows compound 9b or 5b to be obtained selectively by adjusting the conditions.

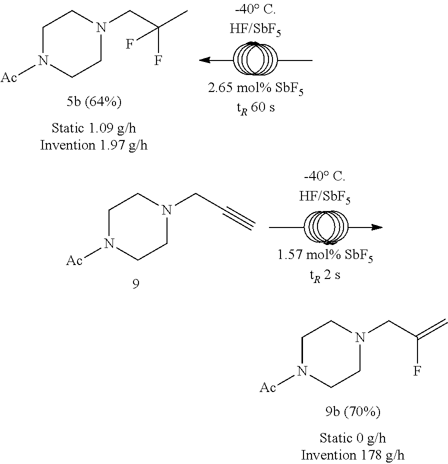

In this example, the production of the fluoro vinyl product 21a of propargylated celecoxib 21 is only possible under the conditions of the invention, which here allows this reaction intermediate, which is inaccessible under batch conditions, to be trapped, as is the case for 9b.

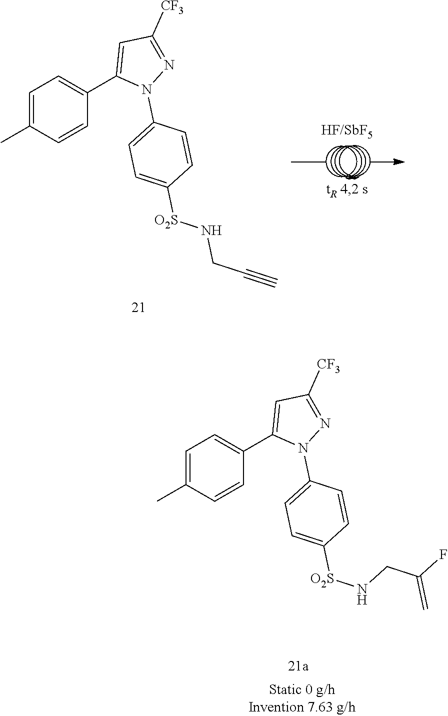

Application of a superacid reagent to compound 5 in static mode produces the difluoro compound 5b and with great difficulty compound 5a, whereas the use of the process of the invention allows compound 5a or 5b to be obtained selectively by adjusting the conditions.

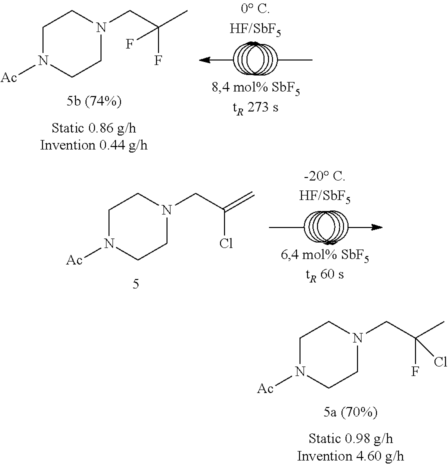

Finally, the facility described in the present invention allows superelectrophilic activation of the celecoxib diallyl derivative 20 and allows tandem cyclization/fluorination reactions producing compound 20a selectively, which is a unit of pharmaceutical interest, with the process of the invention required to assay such acidity (which cannot be achieved under static conditions). Product 20a cannot be obtained under batch conditions with an analogous reaction.

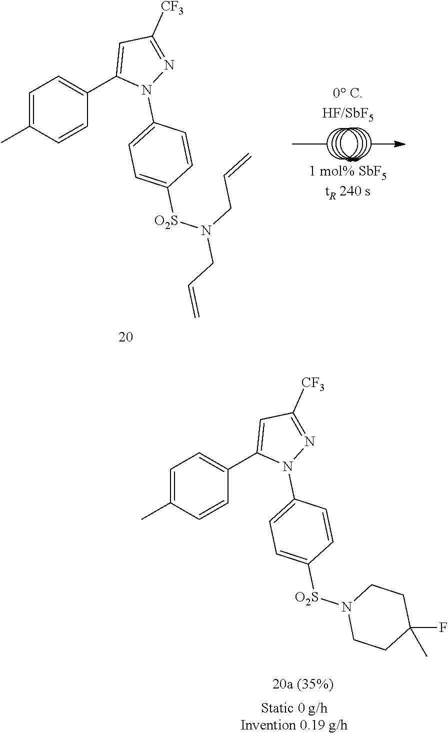 [SMILES: C=CCN(CC=C)S(=O)(=O)c1ccc(-n2nc(C(F)(F)F)cc2-c2ccc(C)cc2)cc1 ; Cc1ccc(-c2cc(C(F)(F)F)nn2-c2ccc(S(=O)(=O)N3CCC(C)(F)CC3)cc2)cc1]

1-13. (canceled)

14. A process for the fluorination and/or cyclization of an amino alkene or alkyne, involving:
a) providing a first phase comprising an amino alkene or alkyne and a second phase comprising a superacid reagent,
b) placing the first and second phases in contact in a continuous-flow microreactor, and
c) recovering the fluorination and/or cyclization product of said amino alkene or alkyne.

15. The process according to claim 14, characterized in that:
the residence time of the first phase and the second phase in the continuous-flow microreactor in step (b) is between 2 seconds and 400 seconds, and/or
the flow rate of the first phase and the flow rate of the second phase in step (b) are independently between 0.1 mL/min and 3.5 mL/min.

16. The process according to claim 14, characterized in that the continuous-flow microreactor comprises a micro-mixer and a tubular pipe, in which the tubular pipe has:
a length of between 20 cm and 800 cm, and
an inside diameter of between 0.5 mm and 2.5 mm.

17. The process according to claim 14, characterized in that it is performed in a facility comprising:
a storage unit for a first phase comprising an amino alkene or alkyne,
a storage unit for a second phase comprising a superacid reagent,
a first phase continuous feed means connected to the first phase storage unit, and to a first phase equilibration tubular pipe,
a second phase continuous feed means connected to the second phase storage unit and to a second phase equilibration tubular pipe,
a continuous-flow microreactor comprising a micro-mixer comprising two inlets and one outlet, and a tubular pipe comprising one inlet and one outlet, in which the inlet of the tubular pipe is connected to the outlet of the micro-mixer,
in which the first phase equilibration tubular pipe is connected to the first inlet of the micro-mixer and the second phase equilibration tubular pipe is connected to the second inlet of the micro-mixer, and
a collection unit, connected to the outlet of the tubular pipe of the microreactor.

18. The process according to claim 14, characterized in that the superacid reagent is selected from the group consisting of HF/MF₅ and HSO₃F/MF₅, where M is Sb, As, P, Ta, or Nb.

19. The process according to claim 14, characterized in that said amino alkene or alkyne is an allyl or propargyl amine.

20. The process according to claim 19, characterized in that said amino alkene or alkyne is an amino alkene of formula (Ia): 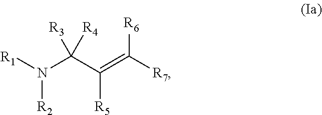
in which:
R₁, R₂, R₃, R₄, R₅, R₆ and R₇ are independently selected from the group consisting of hydrogen, halogen, C₁-C₁₂ alkyl, C₂-C₁₂ alkenyl, C₂-C₁₂ alkynyl, C₁-C₁₂ heteroalkyl, C₃-C₁₂ cycloalkyl, C₂-C₁₂ heterocycloalkyl, aryl, heteroaryl, a group —S(O)₂—R₈ and a group —C(O)—R₉, where R₈ and R₉ are chosen independently from C₁-C₁₂ alkyl, C₂-C₁₂ alkenyl, C₂-C₁₂ alkynyl, C₁-C₁₂ heteroalkyl, C₃-C₁₂ cycloalkyl, C₂-C₁₂ heterocycloalkyl, aryl, and heteroaryl,
said alkyl, alkenyl, alkynyl, heteroalkyl, cycloalkyl, heterocycloalkyl, aryl and heteroaryl groups being optionally substituted, or
two groups each chosen from R₁, R₂, R₃, R₄, R₅, R₆ and R₇ may alternatively form, together with the atom(s) to which they are attached, an optionally substituted 3- to 12-membered ring.

21. The process according to claim 20, characterized in that:
R₁ and R₂ are selected independently from a hydrogen, a C₂-C₁₂ alkenyl, an aryl, optionally substituted with a C₁-C₆ alkyl, a nitro, or a fluoro group selected from the group consisting of —CF₃, —OCF₃, —SCF₃, —OCF₂R′, —OCF(R′)₂, —SCF₂R′ and —SCF(R′)₂ where each R′ independently represents a hydrogen, a halogen, a C₁-C₆ alkyl, a C₂-C₆ alkenyl, a C₂-C₆ alkynyl, C₃-C₆ cycloalkyl, —S(O)₂—R₈ and —C(O)—R₉, where R₈ and R₉ are independently aryl optionally substituted with C₁-C₆ alkyl, nitro or a fluoro group chosen from —CF₃, —OCF₃, —SCF₃, —OCF₂R′, —OCF(R′)₂, —SCF₂R′ and —SCF(R′)₂ where each R′ independently represents hydrogen, halogen, C₁-C₆ alkyl, C₂-C₆ alkenyl, C₂-C₆ alkynyl or a C₃-C₆ cycloalkyl,
or alternatively R₁ and R₂, together with the atom to which they are attached, form a piperidine or piperazine, optionally substituted with an aryl or an acetyl,
R₃ and R₄ are hydrogens, and
R₅, R₆ and R₇ are independently selected from the group consisting of hydrogen and halogen.

22. The process according to claim 19, characterized in that said amino alkene or alkyne is an amino alkyne of formula (Ib): 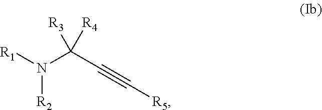
in which:
R₁, R₂, R₃, R₄ and R₅ are independently selected from the group consisting of hydrogen, halogen, C₁-C₁₂ alkyl, C₂-C₁₂ alkenyl, C₂-C₁₂ alkynyl, C₁-C₁₂ heteroalkyl, C₃-C₁₂ cycloalkyl, C₂-C₁₂ heterocycloalkyl, aryl, heteroaryl, a group —S(O)₂—R₈ and a group —C(O)—R₉, where R₈ and R₉ are selected independently from the group consisting of C₁-C₁₂ alkyl, C₂-C₁₂ alkenyl, C₂-C₁₂ alkynyl, C₁-C₁₂ heteroalkyl, C₃-C₁₂ cycloalkyl, C₂-C₁₂ heterocycloalkyl, aryl, and heteroaryl,
said alkyl, alkenyl, alkynyl, heteroalkyl, cycloalkyl, heterocycloalkyl, aryl and heteroaryl groups being optionally substituted, or
two groups each selected from the group consisting of R₁, R₂, R₃, R₄ and R₅ may alternatively form, together with the atom(s) to which they are attached, an optionally substituted 3- to 12-membered ring.

23. The process according to claim 22, characterized in that:
R₁ and R₂ are selected independently from the group consisting of a hydrogen, a C₂-C₁₂ alkenyl, an aryl, optionally substituted with a C₁-C₆ alkyl, a nitro, or a fluoro group chosen from —CF₃, —OCF₃, —SCF₃, —OCF₂R′, —OCF(R′)₂, —SCF₂R′ and —SCF(R′)₂ where each R′ independently represents a hydrogen, a halogen, a C₁-C₆ alkyl, a C₂-C₆ alkenyl, a C₂-C₆ alkynyl or a C₃-C₆ cycloalkyl, —S(O)₂—R₈ and —C(O)—R₉, where R₈ and R₉ are independently aryl optionally substituted with C₁-C₆ alkyl, nitro or a fluoro group selected from the group consisting of —CF₃, —OCF₃, —SCF₃, —OCF₂R′, —OCF(R′)₂, —SCF₂R′ and —SCF(R′)₂ where each R′ independently represents hydrogen, halogen, C₁-C₆ alkyl, C₂-C₆ alkenyl, C₂-C₆ alkynyl or a C₃-C₆ cycloalkyl, or alternatively R₁ and R₂, together with the atom to which they are attached, form a piperidine or piperazine, optionally substituted with an aryl or an acetyl, and
R₃, R₄ and R₅ are hydrogens.

24. The process according to claim 19, characterized in that said amino alkene or alkyne is selected from the group consisting of:

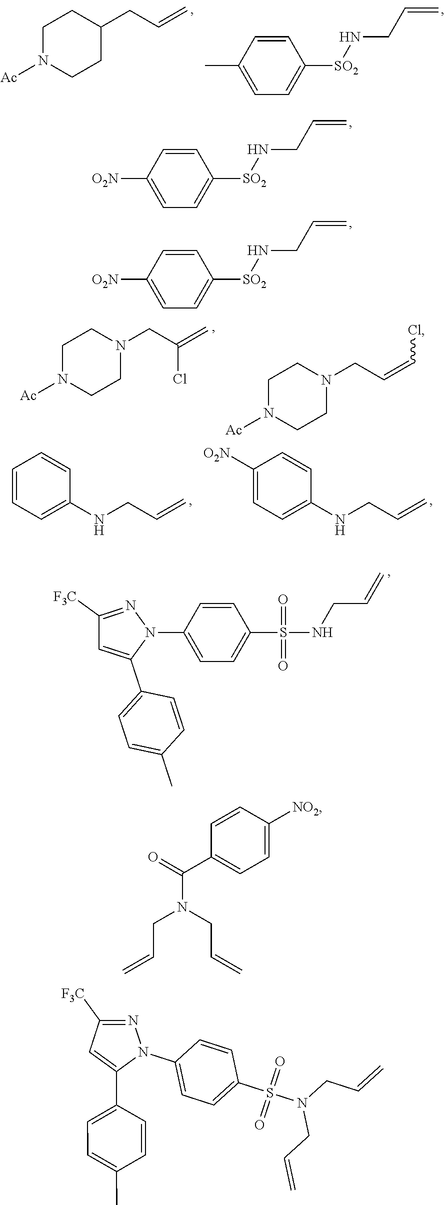

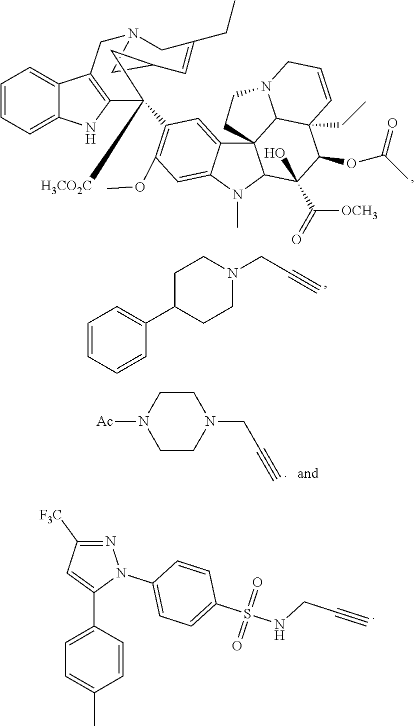

25. A facility for performing the process according to claim 14, characterized in that it comprises:
a storage unit for a first phase comprising an amino alkene or alkyne,
a storage unit for a second phase comprising a superacid reagent,
a first phase continuous feed means connected to the first phase storage unit, and to a first phase equilibration tubular pipe,
a second phase continuous feed means connected to the second phase storage unit and to a second phase equilibration tubular pipe,
a continuous-flow microreactor comprising a micro-mixer comprising two inlets and one outlet, and a tubular pipe comprising one inlet and one outlet, in which the inlet of the tubular pipe is connected to the outlet of the micro-mixer,
in which the first phase equilibration tubular pipe is connected to the first inlet of the micro-mixer and the second phase equilibration tubular pipe is connected to the second inlet of the micro-mixer, and
a collection unit, connected to the outlet of the tubular pipe of the microreactor, in which the tubular pipe of the continuous-flow microreactor has:
a length of between 20 cm and 800 cm, and
an inside diameter of between 0.5 and 2.5 mm.

26. The facility according to claim 25, characterized in that it also comprises:
a 3-way valve providing the connection between the first phase continuous feed means, the first phase storage unit, and the first phase equilibration tubular pipe, and/or
a 3-way valve providing the connection between the second phase continuous feed means, the second phase storage unit and the second phase equilibration tubular pipe.
